(19) United States
(12) Patent Application Publication
(10) Pub. No.: US 2011/0136116 A1
(43) Pub. Date: Jun. 9, 2011

(54) DETECTION OF TARGET NUCLEIC ACID SEQUENCES USING FLUORESCENCE RESONANCE ENERGY TRANSFER

(75) Inventors: Francis Barany, New York, NY (US); Maneesh Pingle, New York, NY (US); Donald Bergstrom, West Lafayette, IN (US)

(73) Assignees: CORNELL UNIVERSITY, Ithaca, NY (US); PERDUE RESEARCH FOUNDATION, West Lafayette, IN (US)

(21) Appl. No.: 12/934,866

(22) PCT Filed: Apr. 8, 2009
(86) PCT No.: PCT/US09/39855

§ 371 Date: Feb. 22, 2011

Related U.S. Application Data
(60) Provisional application No. 61/043,282, filed on Apr. 8, 2008.

(51) Int. Cl. C12Q 1/68; G01N 33/53

(52) U.S. Cl. 435/6.11; 436/94

(57) ABSTRACT

A method for identifying a plurality of target nucleic acid molecules in a sample. The method provides a plurality of oligonucleotide probe sets. Each set comprises a first and a second probe, each having a target-specific portion and a tunable portion with an acceptor or a donor group. The first probe further comprises an endcapped hairpin. A reaction comprises a denaturation and hybridization cycle. Under the hybridization, the set of probes hybridize in a base-specific manner to their respective target nucleotide sequences, and ligate to one another to form a ligation product. Under conditions that permit hybridization of the tunable portions of the ligation product to one another, an internally hybridized ligation product formed, which allows the detection of the fluorescence resonance energy transfer (FRET). A method comprising PCR amplification is also disclosed.

This application claims the benefit of U.S. Provisional Patent Application Ser. No. 61/043,282, filed Apr. 8, 2008, which is hereby incorporated by reference in its entirety.

This invention was made with government support under Public Health Service grant AI062579-03 from the National Institute of Allergy and Infectious Diseases and Grant No. CA65930-08 from the National Cancer Institute. The government has certain rights in this invention.

### FIELD OF THE INVENTION

The present invention relates to the detection of target nucleic acid sequences using fluorescence resonance energy transfer (FRET).

### BACKGROUND OF THE INVENTION

Advances in sequencing technology have led to the proliferation of whole genomic sequence data beginning with the publication of the human genome. The availability of this sequence information has allowed for the design and development of DNA based diagnostic tests for several genetic diseases, e.g. various cancers, cystic fibrosis, etc. In the majority of cases, there is no single marker that provides a definitive diagnosis for cancer; rather assays are designed for a panel of markers. Some markers such as SNPs or mutations only need to be detected as present or absent (Gibson N. J., Clin Chim Acta. 363(1-2): 32-47(2006); Mamotte C. D., Clin Biochem Rev 27(1): 63-75(2006)), while others such as gene expression or copy number variation (Smith et al., Clin Infect Dis 45(8): 1056-1061(2007)), need to be measured quantitatively.

Several pathogenic microorganisms have also been sequenced, including bacteria, fungi, viruses, and protozoa. This has spurred the development of diagnostic assays and tests to detect and identify these organisms in the area of infectious disease. It is also important to identify antibiotic resistance, for example methicillin resistance in Staphylococcus aureus and vancomycin resistance in Enterococcus faecium and Enterococcus faecalis.

Real time and quantitative polymerase chain reaction (“PCR”) based methods are commonly employed in diagnostic tests and assays (Gibson N. J., Clin Chim Acta 363(1-2): 32-47 (2006); Mamotte, C. D. Clin Biochem Rev. 27(1): 63-75(2006); Reynisson et al., J Microbiol Methods 66(2): 206-216(2006); Ruiz-Ponte, C., et al. Clin Chim Acta 363(1-2): 138-146(2006); Kubista et al., Mol Aspects Med. 27(2-3): 95-125(2006)). These include the use of Taqman® probes, molecular beacons, or scorpion probes. A typical Taqman® probe contains a fluorescent reporter at the 5′-end and a moiety at the 3′-end that quenches the fluorescent signal of the reporter. The probe sequence is complementary to the PCR amplicon and is designed to anneal at the extension temperature. During extension, the 5′ to 3′ prime exonuclease activity of Taq DNA polymerase I cleaves the probe, emitting signal due to the separation of the reporter from the quencher. Examples of diagnostic tests that use real time PCR include the Cepheid GeneXpert BCR-ABL test for chronic myelogenous leukemia (“CML”) and Cepheid GeneXpert tests for Group B streptococcus and MRSA (methicillin resistant staphylococcus aureus). Other examples include real time PCR based assays for multiplexed detection of flaviviruses (Dyer et al., J Virol Methods 145(1): 9-13(2007)).

Molecular beacons are similar to Taqman® probes except that the 3′ and 5′ ends of the probe are complementary. When the molecular beacon is free in solution it forms a stem-loop structure which brings the reporter and quencher into immediate proximity of one another. Once hybridized to the amplicon, the stem-loop structure opens, resulting in fluorescent signal. Molecular beacon based assays have been used for pathogen detection, e.g. SARS coronavirus (Horejsh et al., Nucleic Acids Res. 33(2): e13 (2005)).

Scorpion probes incorporate the primer and probe in a single stem-loop oligonucleotide. A portion of the probe primes the amplification and another portion of the probe binds to the newly generated amplicon, this distances a fluorophore on the probe from the quencher, resulting in an increase in fluorescence. Scorpion probes have been used to detect protozoan pathogens such as Giardia and Cryptosporidium (Ng et al., J Clin Microbial 43(3): 1256-1260 (2005) and Stroup et al., J Med Microbial. 55(9): 1217-1222(2006)).

Multiplexed PCR/LDR (ligase detection reaction) has been used for genotyping (Kirk et al., Nucleic Acids Res. 30(15): 3295-3311(2002)) as well as for pathogen detection and identification. Bacterial identification using PCR/LDR relies on multiplexed amplification of regions of the 16s ribosomal RNA gene using universal primers. Specific SNPs in these amplicons are then queried in a multiplexed LDR with the probes designed to provide a hierarchical readout. Certain SNPs are used to distinguish gram positive bacterial from gram negative bacteria; other SNPs classify the organism as belonging to one or more genera, while subsequent SNPs identify the organism to species.

Real time PCR and molecular beacons have many advantages. These include ease of use, avoiding cross-over contamination and providing a quantitative readout. A disadvantage of these methods, however, is their lack of multiplexing capability as they are limited to simultaneous use of 4-6 dyes, of which one is an internal control. Thus, at most, 4 to 6 items can be multiplexed at once.

LDR on the other hand is amenable to a high degree of multiplexing. In a standard LDR reaction, a pair of probes hybridize to a template adjacent to each other. If there is perfect complementarity at the junction, a high fidelity ligase enzyme ligates the probes. One of the LDR probes is labeled with a fluorescent dye to enable the ligation product to be detected. The resulting LDR products can then be separated by (gel or capillary) electrophoresis as a function of their length, or by hybridizing the ligation products to zipcodes on a universal array through complementary zipcodes appended to the end of one of the LDR probes. The level of multiplexing can be increased by using multiple color dyes. However, both the electrophoresis and universal array methods for separating and detecting the ligation products require an additional step that must be performed after the LDR reaction using additional equipment.

Single pair fluorescence resonance energy transfer (FRET)-LDR (spFRET-LDR) is a variation of LDR where the upstream and downstream probes have complementary tails attached to them. The upstream probe has a donor dye while the downstream probe has an acceptor dye. When a ligation product is formed, it is denatured from the template and rehybridizes to form a panhandle structure due to the complimentary tails attached to the upstream and downstream probes. This brings the donor and acceptor in close proximity, allowing for a FRET signal when excited at the appropriate wavelength. (Wabuyele et al., J Am Chem Soc 125(23): 6937-6945(2003)).

This approach is based on the fact that two complementary DNA sequences will hybridize with far greater avidity if they are tethered to each other, since this greatly increases the local concentration of the two probes. In other words, when the two probes are attached to each other, the resultant product has a significantly higher melting temperature (Tm) value across the complementary region, and, consequently, the FRET signal will be maintained even at a higher temperature. As an example, if the complementary region consists of two 10 base sequences, the Tm value of the two individual complementary sequences (as tails on LDR probes) may range from approximately 30° C. to 50° C., while the Tm value of the two individual complementary sequences (as products of the LDR reaction) may range from approximately 70° C. to 90° C. By reading the FRET signal at a temperature significantly above the Tm of the unligated sequences, but below or approximately at the Tm of the ligated sequence (i.e. from 60° C. to 90° C.), one can readily distinguish the ligated products from the unligated products.

spFRET-LDR is not ideal for obtaining a multiplexed readout signal, wherein the same donor and acceptor molecules are used to provide readouts at different temperatures. The melting temperature (Tm) of a spFRET-LDR product panhandle will be dependent on both the length and the particular bases of the intervening sequence. If the intervening sequence transiently creates its own secondary structure, such a structure may enhance formation of the panhandle, thus increasing the Tm of the LDR product. If the intervening sequence is AT rich and very long, such a sequence may slow down formation of the panhandle, thus decreasing the Tm of the LDR product. For these reasons, it is difficult to design a set of LDR probes having the same acceptor and donor groups to different targets, where two or more LDR product signals may be readily distinguished through their different Tm values. Further, such design efforts would need to be repeated with each set of new targets.

The present invention is directed to overcoming these and other deficiencies in the art

### SUMMARY OF THE INVENTION

The present invention is directed to a method for identifying one or more of a plurality of target nucleic acid molecules in a sample. This method includes providing a sample potentially containing one or more target nucleic acid molecules and a plurality of oligonucleotide probe sets. Each probe set is characterized by (a) a first oligonucleotide probe, having a target-specific portion and a tunable portion with an endcapped hairpin and (b) a second oligonucleotide probe having a target specific portion and a tunable portion, wherein one of the first and second oligonucleotide probes has an acceptor group and the other of the first and second probes has a donor group. A ligase is provided and blended with the sample and the plurality of oligonucleotide probe sets to form a ligase detection reaction mixture. The mixture is subjected to one or more ligase detection reaction cycles with each cycle comprising a denaturation and hybridization treatment. During the denaturation treatment, any hybridized oligonucleotides are separated from the target nucleic acid sequences, and, during the hybridization treatment, the set of oligonucleotide probes hybridize in a base-specific manner to their respective target nucleotide sequences, if present in the sample, and ligate to one another to form a ligation product. The ligation product contains the tunable portions, the endcapped hairpin, the target-specific portions, the acceptor group, and the donor group. The ligation products are subjected to conditions effective to permit hybridization of the tunable portions of the ligation product to one another to form an internally hybridized ligation product. The fluorescence resonance energy transfer (FRET) between the donor and acceptor groups of the internally hybridized ligation product is detected, thereby indicating the presence of a target nucleic acid molecule in the sample.

In a second aspect of the present invention, a sample potentially containing one or more target nucleic acid molecules and a plurality of oligonucleotide primer sets are provided. Each primer of a primer set comprises a target-portion and a universal tail portion. A DNA polymerase is also provided and the sample, the plurality of oligonucleotide primers, and the polymerase are blended to form a polymerase chain reaction (PCR) mixture. The PCR mixture is subjected to one or more polymerase chain reaction cycles. Each cycle includes a denaturation treatment, where hybridized nucleic acid sequences are separated; a hybridization treatment, where the primers hybridize to their complementary target-specific portions; and an extension treatment, where the hybridized primers are extended to form extension products. A plurality of oligonucleotide probe sets is also provided. Each probe set is characterized by (a) a first oligonucleotide probe, having a target-specific portion and a tunable portion with an endcapped hairpin and (b) a second oligonucleotide probe having a target specific portion and a tunable portion, wherein one of the first and second oligonucleotide probes has an acceptor group and the other of the first and second probes has a donor group. A ligase is provided, and blended with the sample containing the extension products and the plurality of oligonucleotide probe sets to form a ligase detection reaction mixture. The mixture is subjected to one or more ligase detection reaction cycles, each cycle comprising a denaturation and hybridization treatment. During the denaturation treatment, any hybridized oligonucleotides are separated from the target nucleic acid sequences. During the hybridization treatment, the set of oligonucleotide probes hybridize in a base-specific manner to their respective target nucleotide sequences, if present in the sample, and ligate to one another to form a ligation product containing the tunable portions, the endcapped hairpin, the target-specific portions, the acceptor group, and the donor group. The ligation products are subjected to conditions effective to permit hybridization of the tunable portions of the ligation product to one another to form an internally hybridized ligation product. The fluorescence resonance energy transfer (FRET) between the donor and acceptor groups of the internally hybridized ligation product is detected, thereby indicating the presence of a target nucleic acid molecule in the sample.

Another aspect of the present invention involves a kit for identifying one or more of a plurality of target nucleic acid molecules in a sample. This kit includes a ligase and a plurality of oligonucleotide probe sets. Each probe set is characterized by (a) a first oligonucleotide probe comprising a target-specific portion and a tunable portion with an endcapped hairpin and (b) a second oligonucleotide probe comprising a target specific portion and a tunable portion, wherein one of the first and second oligonucleotide probes has an acceptor group and the other of the first and second probes has a donor group.

Another aspect of the present invention is directed to a method for detecting one or more of a plurality of target nucleic acid molecules in a sample. This method includes providing a sample potentially containing one or more target nucleic acid molecules and a plurality of oligonucleotide probe sets. Each probe set is characterized by a first oligonucleotide probe having a target-specific portion and a tunable portion with an endcapped hairpin and a second oligonucleotide probe having a target specific portion and a tunable portion, wherein one of the first and second oligonucleotide probes has an acceptor group and the other of the first and second probes has a donor group. The nucleotide sequence of the tunable portion of the first oligonucleotide probe in a probe set is complementary to the nucleotide sequence of the tunable portion of the second oligonucleotide probe in a probe set. The sample and the plurality of oligonucleotide probe sets are blended to form a hybridization mixture. The mixture is subjected to one or more hybridization cycles, each cycle involving a denaturation and hybridization treatment. During the denaturation treatment, any hybridized nucleic acid sequences are separated. During the hybridization treatment, the target-specific portions of a set of oligonucleotide probes hybridize to their respective target nucleotide sequences, if present in the sample, and the tunable portions of the set of oligonucleotide probes hybridize to each other to form an internally hybridized oligonucleotide probe set. The fluorescence resonance energy transfer (FRET) between the donor and acceptor groups of each internally hybridized oligonucleotide probe set is detected, thereby indicating the presence of a target nucleic acid sequence in the sample.

A final aspect of the present invention is directed to a kit for detecting one or more of a plurality of target nucleic acid molecules in a sample. This kit includes a plurality of oligonucleotide probe sets. Each probe set is characterized by a first oligonucleotide probe comprising a target-specific portion and a tunable portion with an endcapped hairpin, and a second oligonucleotide probe having a target specific portion and a tunable portion, wherein one of the first and second oligonucleotide probes has an acceptor group and the other of the first and second probes has a donor group.

The primary advantage of the present invention is the utilization of oligonucleotide probes bearing tunable portions that include a tunable endcapped hairpin, which allows for the donor and acceptor fluorophores to undergo FRET at predefined melting temperatures. Since the melting temperature (“Tm”) of the ligation product hairpin is controlled by the sequence of the approximately 4 to 6 perfectly complementary bases adjacent to, as well as the structure of the endcapped sequence, it is independent of the LDR probe sequences as well as independent of the LDR product sequence length. Further, it is distinguished by having a tight melting profile. Thus, the endcapped design overcomes the deficiencies of spFRET-LDR probes. The tunable endcapped hairpin approach allows for design of a standardized set of sequences which may be appended to LDR probes, giving reliable and predictable Tm values that are easily distinguishable in a multiplexed format, independent of the target sequence being probed. When coupled to LDR assays, the probes of the present invention enable multiplexing of between 48-60 signals. Assays can be performed on standard real time PCR instrumentation, such as the ABI 7500 or the Cepheid GeneXpert system, which enables both thermal cycling for the LDR as well as readout of the signals. When used for real time assays analogous to Taqman® probes, the oligonucleotide probes of the present invention allow for multiplexing of up to 18 targets simultaneously.

### BRIEF DESCRIPTION OF THE DRAWINGS

FIG. 1 shows one structural embodiment of the oligonucleotide probes bearing donor (D) and acceptor (A) fluorophores that undergo FRET (fluorescence resonance energy transfer) in accordance with the present invention.

FIG. 2 demonstrates the signal specificity achieved using the oligonucleotide probes of the present invention.

FIG. 3 depicts one structural embodiment of the oligonucleotide probes bearing donor (D) and acceptor (A) fluorophores that undergo real-time FRET detection in accordance with the present invention.

FIG. 4 shows an example of the DNA interstrand distance of a standard purine-pyrimidine base pair.

FIGS. 5A-B show the structure of two aromatic endcap molecules (FIG. 5A) and how each of the aromatic endcap molecules attach to the and 5′ termini of a pair of complementary oligonucleotides (FIG. 5B) in accordance with the present invention.

FIGS. 6A-B show the structure of four different aliphatic endcap molecules (FIG. 6A) and how each of the aliphatic endcap molecules attach to the 3′- and 5′ termini of a pair of complementary oligonucleotides (FIG. 6B) in accordance with the present invention.

FIG. 7 is a schematic representation of a multiplexed PCR/LDR assay utilizing oligonucleotide probe-FRET readout for identification of pathogens in accordance with the present invention.

FIG. 8 is an illustration of oligonucleotide probes designed to provide readout for nine different signals using six different donor-acceptor FRET pairs.

FIG. 9 depicts a representative melting curve for the detection of three distinct ligation products with probes tuned to melt at 60, 70, and 80° C. The ligation product containing the probe with the lowest melting temperature is produced in 3× excess of the ligation product containing the probe with the highest melting temperature. The melting curve (yellow line) in this example is generated by slowly cooling the temperature from 100° C. to 40° C. (blue line). The red curve represents the derivative of the melting curve.

FIG. 10 depicts a representative melting curve for the detection of three distinct ligation products with probes tuned to melt at 60, 70, and 80° C. The ligation product containing the probe with the highest melting temperature is produced in 3× excess of the ligation product containing the probe with the lowest melting temperature. The melting curve (yellow line) in this example is generated by slowly cooling the temperature from 100° C. to 40° C. (blue line). The red curve represents the derivative of the melting curve.

FIG. 11 shows a representative melting curve for the detection of three distinct ligation products with probes tuned to melt at 60, 70, and 80° C. The ligation product containing the probe with the lowest melting temperature is produced in 3× excess of the highest melting temperature. The melting curve (yellow line) in this example is generated by slowly raising the temperature from 40° C. to 100° C. (blue line). The red curve represents the derivative of the melting curve.

FIG. 12 shows a representative melting curve for the detection of three distinct ligation products with probes tuned to melt at 60, 70, and 80° C. The ligation product containing the probe with the highest melting temperature is produced in 3× excess of the product containing the probe with the lowest melting temperature. The melting curve (yellow line) in this example is generated by slowly raising the temperature from 40° C. to 100° C. (blue line). The red curve represents the derivative of the melting curve.

FIG. 13 is a typical melting curve, displaying the peaks obtained for homozygous and heterozygous SNPs detected by probe FRET. In this illustration, SNP1 is homozygous for allele B and produces a signal at 60° C. SNP2 is heterozygous for both alleles and results in a peak that has a different shape as it is a combination of two separate peaks, one for allele A at 76° C. and the other for allele B at 80° C. The melting curve (yellow line) in this example is generated by slowly raising the temperature from 40° C. to 100° C. (blue line). The red curve represents the derivative of the melting curve.

FIG. 14 is a typical melting curve, displaying the peaks obtained for homozygous SNPs detected by probe FRET. In this illustration, SNP1 is homozygous for allele A (peak at 56° C.) and SNP 2 is homozygous for allele A (peak at 76° C.). The melting curve (yellow line) in this example is generated by slowly raising the temperature from 40° C. to 100° C. (blue line). The red curve represents the derivative of the melting curve.

FIG. 15 is a typical melting curve displaying the peaks obtained for four different SNPs (one belonging to each of the four amplicon groups) detected using a single donor-acceptor FRET pair. The presence of one or both alleles is determined by looking at the derivative curve (red curve). The Group A SNPs are read between 54-62° C., Group B between 64-72° C., Group C between 74-82° C., and Group D between 84-92° C. The melting curve (yellow line) is generated by slowly raising the temperature from 40° C. to 100° C. (blue line) and reading the FRET signals. Therefore, the lower melting probe products (Group A) are read first, followed by Group B, Group C, and Group D.

FIG. 16 is a typical melting curve, displaying the peaks obtained for four different SNPs detected using a single donor-acceptor FRET pair as in FIG. 15.

FIG. 17 is a typical melting curve, displaying the peaks obtained for four different SNPs detected using a single donor-acceptor FRET pair as in FIG. 15.

FIG. 18 is a typical melting curve, displaying the peaks obtained for four different SNPs detected using a single donor-acceptor FRET pair as in FIG. 15.

FIG. 19 is a typical melting curve, displaying the peaks obtained for homozygous and heterozygous SNPs detected by probe-FRET. In this illustration, SNP1 is homozygous for allele B and produces a signal at 60° C. SNP2 is heterozygous for both alleles and results in a peak that has a different shape as it is a combination of two separate peaks, one for allele A at 76° C. and the other for allele B at 80° C. The yellow line represents the melt curve. The derivative of the melt curve is displayed in red. The melting curve in this example is generated by slowly cooling the temperature from 100° C. to 40° C. (blue line).

FIG. 20 is a typical melting curve, displaying the peaks obtained for homozygous SNPs detected by probe FRET. The yellow line represents the melt curve. The derivative of the melt curve is displayed in red. The blue line shows the temperature ramp while reading the fluorescence from the FRET. In this illustration, SNP1 is homozygous for allele A (peak at 56° C.) and SNP 2 is homozygous for allele A (peak at 76° C.).

FIG. 21 is a typical melting curve, displaying the peaks obtained for four different SNPs (one belonging to each of the four amplicons) detected using a single donor-acceptor FRET pair, The Group A SNPs are read between 84-92° C., Group B between 74-82° C., Group C between 64-72° C., and Group D between 54-62° C. The melting curve (yellow line) is measured by heating the ligation products to 100° C. and then slowly cooling to 40° C. (blue line) to read the FRET signals. Therefore, the higher melting probe products (Group A) are read first, followed by Group B, Group C, and Group D.

FIG. 22 is a typical melting curve, displaying the peaks obtained for four different SNPs detected using a single donor-acceptor FRET pair, as in FIG. 21.

FIG. 23 is a typical melting curve, displaying the peaks obtained for four different SNPs detected using a single donor-acceptor FRET pair, as in FIG. 21.

FIG. 24 is a typical melting curve, displaying the peaks obtained for four different SNPs detected using a single donor-acceptor FRET pair as, in FIG. 21

FIG. 25 shows typical melting curves generated using probes having two different donor-acceptor pairs (multiplex-LDR). Using two different donor-acceptor pairs allows for the detection of two sets of SNPs in each group of PCR amplicons. After five rounds of LDR, the reaction is heated to 100° C. to completely denature the ligation products from the template and slowly cooled to measure the fluorescence from both FRET donor-acceptor pairs. The light blue line shows the temperature ramp and traces the LDR cycling conditions. The purple and green lines to the left of each melting temperature range shown on the x-axis represent the increasing fluorescence signal accumulated from all the ligation products bearing that donor-acceptor pair. The green and purple lines corresponding to the melting temperature range for detection show the melt curve measured for the two donor-acceptor FRET pairs. The red line represents the derivative of the green melt curve, while the yellow line represents the derivative of the purple melt curve.

FIG. 26 shows typical melting curves generated using probes having two different donor-acceptor pairs (multiplex-LDR). Using two different donor-acceptor pairs allows for the detection of two sets of SNPs in each group of PCR amplicons. After five rounds of LDR, the reaction is heated to 100° C. to completely denature the ligation products from the template and slowly cooled to measure the fluorescence from both FRET donor-acceptor pairs. At the top left of the graph, the light blue line traces the LDR cycling conditions and the temperature ramp. At the bottom left of the graph, the purple and green lines represent the increasing fluorescence signal accumulated from all the ligation products bearing that donor-acceptor pair. On the right hand side of the graph, the green and purple lines show the melt curve measured for the two donor-acceptor FRET pairs. The red line represents the derivative of the green melt curve, while the yellow line represents the derivative of the purple melt curve.

FIG. 27 shows melting curves generated using probes with three distinct donor-acceptor FRET pairs. After five rounds of LDR, the reaction was heated to 100° C. to completely denature the ligation products from the template and slowly cooled to measure the FRET signal. At the top left of the graph, the light blue line traces the LDR cycling conditions and the temperature ramp. At the bottom left of the graph, the purple, brown, and orange lines represent the increasing fluorescence signal accumulated from all the ligation products bearing that donor-acceptor pair. On the right hand side of the graph, the purple, brown, and orange lines show the melt curves measured for the three donor-acceptor FRET pairs. The red line represents the derivative of the purple melt curve; the green line represents the derivative of the brown melt curve, while the yellow line represents the derivative of the orange melt curve.

FIGS. 28A-B are schematic representations of multiplex PCR/LDR using oligonucleotide probes with FRET detection for genotyping, in accordance with the present invention.

FIG. 29 shows a typical melting curve generated using the multiplex PCR/LDR method with two distinct donor-acceptor FRET pairs.

FIG. 30 shows a typical melting curve generated using the multiplex PCR/LDR method with two distinct donor-acceptor FRET pairs.

FIG. 31 shows a typical melting curve generated using the multiplex PCR/LDR method with three distinct donor-acceptor FRET pairs.

FIG. 32 shows a typical melting curve generated using the multiplex PCR/LDR method with three distinct donor-acceptor FRET pairs.

FIG. 33 shows a typical melting curve generated using the multiplex PCR/LDR method with three distinct donor-acceptor FRET pairs

FIG. 34 shows a typical melting curve generated using the multiplex PCR/LDR method with three distinct donor-acceptor FRET pairs.

FIG. 35 shows the use of the oligonucleotide probes with FRET detection for real-time quantitative measure gene expression.

FIG. 36 shows relative gene expression levels measured using real-time probes. The red line at the top traces the cycling conditions during the reaction. The fastest amplifying targets (group A) have cycle threshold (“Ct”) values between 14 and 20 cycles (threshold denoted by dotted red line). Group B amplicons have Ct values between 20 and 28 (threshold denoted by dotted grey line), while Group C amplicons have Ct values higher than 28 (threshold denoted by dotted black line). The threshold signal for each group is higher than the previous group, because the signal for a particular color is accumulating for all three groups from the first cycle.

### DETAILED DESCRIPTION OF THE INVENTION

The present invention is directed to a method for identifying one or more of a plurality of target nucleic acid molecules in a sample. This method includes providing a sample potentially containing one or more target nucleic acid molecules and a plurality of oligonucleotide probe sets. Each probe set is characterized by (a) a first oligonucleotide probe, having a target-specific portion and a tunable portion with an endcapped hairpin and (b) a second oligonucleotide probe having a target specific portion and a tunable portion, wherein one of the first and second oligonucleotide probes has an acceptor group and the other of the first and second probes has a donor group. A ligase is provided and blended with the sample and the plurality of oligonucleotide probe sets to form a ligase detection reaction mixture. The mixture is subjected to one or more ligase detection reaction cycles with each cycle comprising a denaturation and hybridization treatment. During the denaturation treatment any hybridized oligonucleotides are separated from the target nucleic acid sequences, and, during the hybridization treatment, the set of oligonucleotide probes hybridize in a base-specific manner to their respective target nucleotide sequences, if present in the sample, and ligate to one another to form a ligation product. The ligation product contains the tunable portions, the endcapped hairpin, the target-specific portions, the acceptor group, and the donor group. The ligation products are subjected to conditions effective to permit hybridization of the tunable portions of the ligation product to one another to form an internally hybridized ligation product. The fluorescence resonance energy transfer (FRET) between the donor and acceptor groups of the internally hybridized ligation product is detected, thereby indicating the presence of a target nucleic acid molecule in the sample.

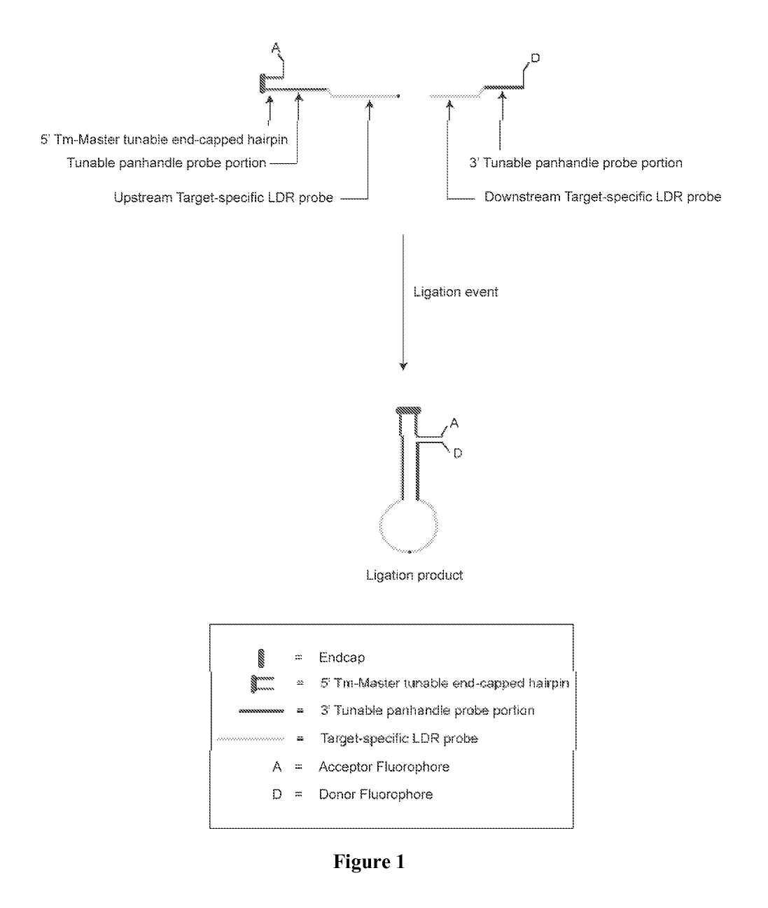

FIG. 1 shows a preferred embodiment of the structure of the oligonucleotide probes of this aspect of the present invention. In this embodiment, the first oligonucleotide probe of a probe set comprises a 3′ target-specific region, a tunable panhandle probe portion, and a 5′ master Tm tunable end-capped hairpin with an acceptor molecule (A). The second oligonucleotide probe of a probe set comprises a 5′ target-specific portion and a 3′ tunable panhandle probe portion with a donor molecule (D). In an alternative embodiment, the first oligonucleotide probe of a probe set comprises a 3′ target-specific region, a tunable panhandle probe portion, and a 5′ master Tm tunable end-capped hairpin with a donor molecule and the second oligonucleotide comprises a 5′ target-specific region and a 3′ tunable panhandle probe portion with an acceptor molecule. In another embodiment, the first oligonucleotide probe of a probe set comprises a 3′ target specific region and a 5′ tunable portion with a donor or acceptor molecule, and the second oligonucleotide probe of a probe set comprises a 5′ target-specific region, a tunable panhandle portion, and a 3′ master Tm tunable end-capped hairpin with an acceptor molecule or with the donor molecule. Consistent with all of the above described probe designs, the donor D and acceptor A fluorophores undergo FRET (fluorescence resonance energy transfer) when they are in close proximity to each other. In accordance with the present invention, this occurs when a ligation event, followed by internal hybridization of the 5′ and 3′ tunable portions of the ligation products, takes place.

The oligonucleotide probe sets can be in the form of ribonucleotides, deoxynucleotides, modified ribonucleotides, modified deoxyribonucleotides, peptide nucleotide analogues, modified peptide nucleotide analogues, modified phosphate-sugar-backbone oligonucleotides, nucleotide analogs, and mixtures thereof.

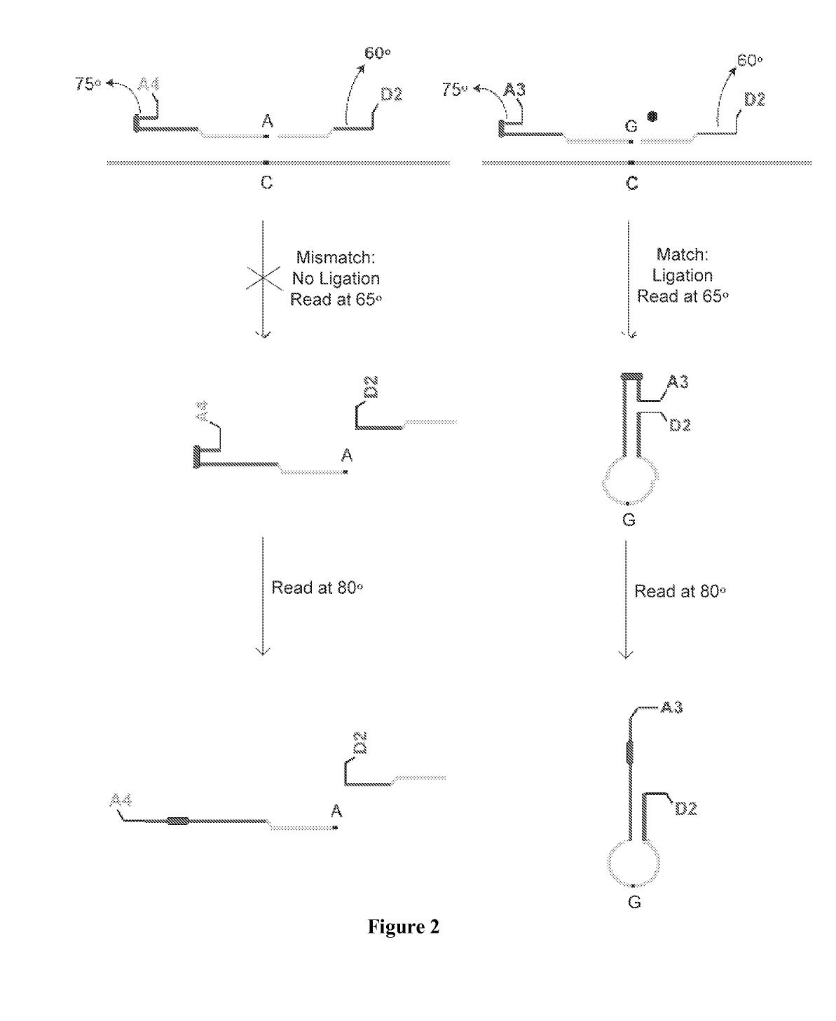

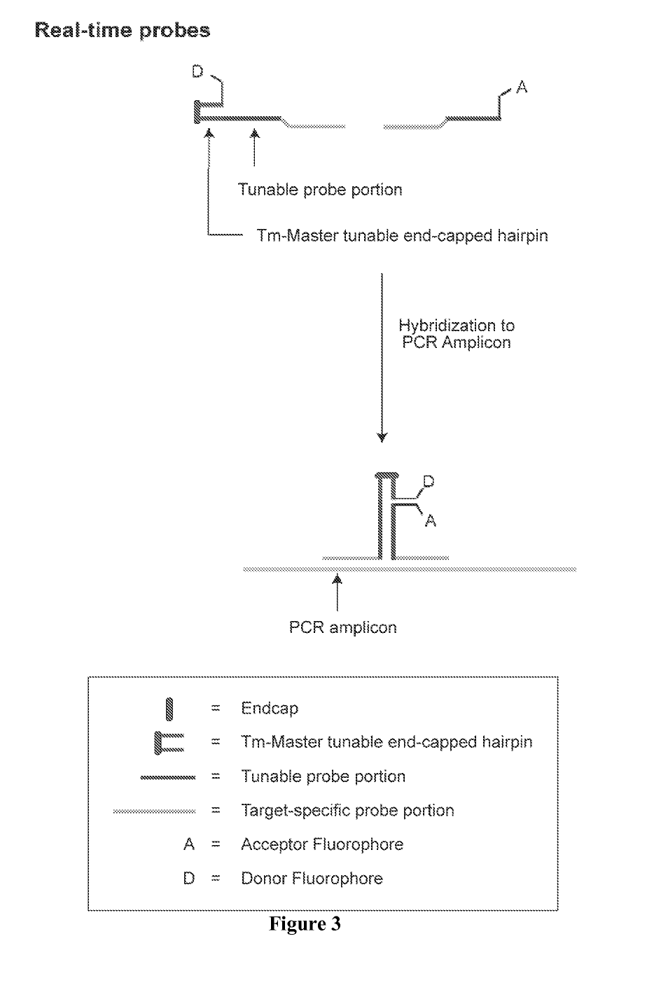

As shown in FIG. 2, the oligonucleotide probe sets are suitable for ligation together at a ligation junction when hybridized adjacent to one another on a corresponding target nucleotide sequence due to perfect complementarity at the ligation junction. However, when the oligonucleotide probe sets are hybridized to any other nucleotide sequence present in the sample, a mismatch at a base at the ligation junction interferes with such ligation.

After ligation occurs, the ligation product is denatured from the template and the tunable portions internally hybridize so that a loop forms at one end (comprised of the target specific portions of the probe), an intervening stem (the tunable probe portions of the probes) and the end-capped hairpin at the other end (See FIG. 2). The tunable portion with the endcapped hairpin of the probes are designed to have predefined melting temperatures (“Tm”) with tight melting transitions such that a given probe melts completely over approximately 5-10° C. Melting temperatures for the probes can be individually tuned through the use of end-capped hairpins and preselected oligonucleotide sequences. The probe Tm is a function of the endcap, the stem length, and the GC content and order of the nucleotides in the stem.

When the ligation product is fully formed, the donor and acceptor fluorophores are brought in proximity to each other, resulting in a FRET at the predefined melting temperature controlled by the tunable portions and the endcapped hairpin. If the temperature is raised above the tuned Tm value of the ligation product, the end-capped hairpin melts open, the tunable probe portions also melt, and the donor and acceptor dyes are no longer in close proximity (FIG. 2). As a result, there is no FRET signal. Likewise, if there is no ligation during the LDR (i.e. correct target is not present), the ligation product cannot form and there is no FRET signal as also shown in FIG. 2.

The ligase detection reaction process used in the methods of the present invention, is well known in the art and described generally in WO 90/17239 to Barany et al., Barany et al., “Cloning, Overexpression and Nucleotide Sequence of a Thermostable DNA Ligase-encoding Gene,” Gene, 109:1-11 (1991), and Barany F., “Genetic Disease Detection and DNA Amplification Using Cloned Thermostable Ligase,” Proc. Natl. Acad. Sci. USA, 88:189-193 (1991), which are hereby incorporated by reference in their entirety. In accordance with the present invention, the ligase detection reaction can use two sets of complementary oligonucleotides. This is known as the ligase chain reaction which is described in the three immediately preceding references, which are hereby incorporated by reference in their entirety. Alternatively, the ligase detection reaction can involve a single cycle which is known as the oligonucleotide ligation assay (see Landegren et al., “A Ligase-Mediated Gene Detection Technique,” Science 241:1077-80 (1988); Landegren et al., “DNA Diagnostics—Molecular Techniques and Automation,” Science 242:229-37 (1988); and U.S. Pat. No. 4,988,617 to Landegren et al. which are hereby incorporated by reference in their entirety).

During the ligase detection reaction phase of the present invention, the denaturation treatment is carried out at a temperature of 80-105° C., while hybridization takes place at 50-85° C., depending on the melting temperature of the target specific portions of the oligonucleotide probes. Each cycle comprises a denaturation treatment and a thermal hybridization treatment which in total is from about one to five minutes long. Typically, the ligation detection reaction involves repeatedly denaturing and hybridizing for 2 to 50 cycles. The total time for the ligase detection reaction phase of the process is 1 to 250 minutes.

Preferably, the ligase used in the ligase detection reaction is a thermostable ligase, such as that derived from Thermus aquaticus. This enzyme can be isolated as described by Takahashi et al., “Thermophillic DNA Ligase,” J. Biol. Chem. 259:10041-47 (1984), which is hereby incorporated by reference in its entirety, or prepared recombinantly. Procedures for isolation and the recombinant production of Thermus aquaticus ligase as well as Thermus themophilus ligase are disclosed in WO 90/17239 to Barany et al., and Barany et al., “Cloning, Overexpression and Nucleotide Sequence of a Thermostable DNA-Ligase Encoding Gene,” Gene 109:1-11 (1991), which are hereby incorporated by reference in their entirety. These references contain complete sequence information for this ligase as well as the encoding DNA. Other suitable ligases include E. coli ligase, T4 ligase, Thermus sp. AK16 ligase, Aquifex aeolicus ligase, Thermotoga maritima ligase, and Pyrococcus ligase.

The ligation amplification mixture may include a carrier DNA, such as salmon sperm DNA.

Multiple allele differences at one or more nucleotide positions in a single target nucleic acid molecule or multiple allele differences at one or more positions in multiple target nucleic acid molecules can be distinguished using the methods of the present invention. The oligonucleotide probe sets form a plurality of oligonucleotide probe groups. Each group is comprised of one or more oligonucleotide probe sets designed for distinguishing multiple allele differences at a single nucleotide position. In each group, the second oligonucleotide probe of each probe set has a common target-specific portion, and the first oligonucleotide probe of each probe set has differing target-specific portions which hybridize to a given allele in a base-specific manner. The first oligonucleotide probes also have differing endcapped hairpin tunable probe portions which allow for the differential detection of FRET signals of internally hybridized ligation products from each probe set within each probe group. Detection of the FRET signal indicates the presence of one or more alleles at one or more nucleotide positions in one or more target nucleotide sequences in the sample.

The one or more first oligonucleotide probes in a probe group have endcapped hairpin tunable portions with differing melting temperatures. In a preferred embodiment, the melting temperatures of the endcapped hairpin tunable portions of the first oligonucleotide probes in a probe group differ by at least 4° C. The endcapped hairpin tunable portions of first oligonucleotide probes in a probe group which have differing melting temperatures may have the same acceptor group. Alternatively, the endcapped hairpin tunable portions of first oligonucleotide probes in a probe group having differing melting temperatures may have different acceptor groups.

The melting temperature of the target-specific portions of the oligonucleotide probes in a probe group is determined by the target nucleotide sequence to be detected and will typically range between 55-80° C. The melting temperature of the target-specific portion differs from the melting temperature of the tunable portions of the oligonucleotide probes in a probe group. The melting temperature of the target-specific portion can be higher or lower than the melting temperature of the tunable portions of the oligonucleotide probes in a probe group.

The endcapped hairpin tunable portion of the one or more first oligonucleotide probes in a first probe group may have a melting temperature which differs from the endcapped hairpin tunable portion of one or more first oligonucleotide probes in a second probe group. In a preferred embodiment, the melting temperatures of the endcapped hairpin tunable portions differ by at least 6° C. from one probe group to the next probe group. In an alternative embodiment, probe groups may have one or more first oligonucleotide probes with similar endcapped hairpin tunable portion melting temperatures. In this embodiment, the probe groups have different acceptor-donor groups.

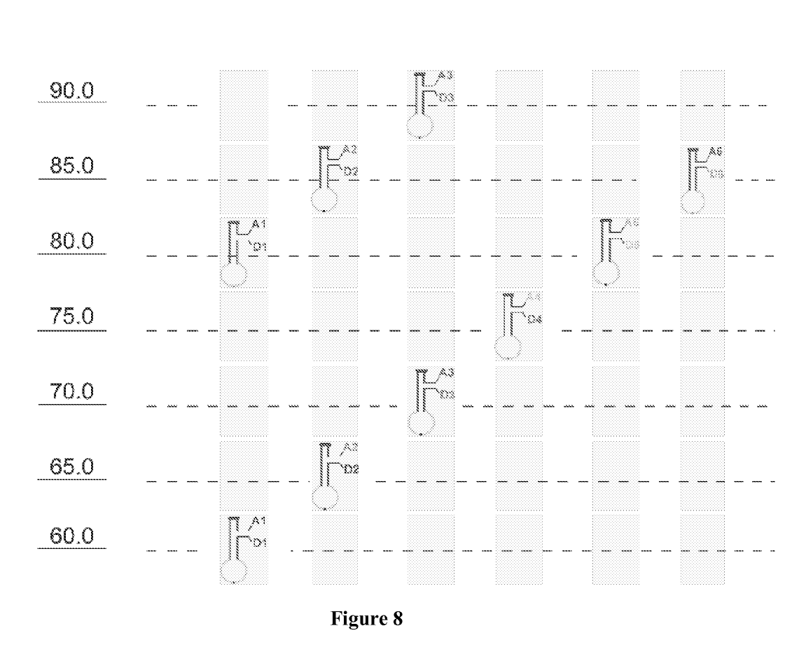

The plurality of internally hybridized ligation products are detected by performing a melt curve analysis and detecting FRET between the donor and acceptor molecules. The plurality of internally hybridized ligation products can be detected at one or more FRET signals. Ligation products with the same FRET signals can be distinguished by their melting peaks on a first derivative of the melt curve. The melting peaks of the derivative curve correspond to the melting temperatures of the tunable portions of the ligation products. In one embodiment, the same FRET signals from ligation products within the same probe group are distinguished by the melting peaks of a first derivative of the melt curve which differ by at least 4° C. In another embodiment, the same FRET signals from ligation products from different probe groups are distinguished by the melting peaks of a first derivative of the melt curve, which differ by at least 6° C. FIG. 8 provides an example of how ligation probes may be designed to provide a readout with nine different signals using six different donor-acceptor FRET pairs. In this example, two ligation products are detected with probes bearing the donor-acceptor FRET pair D1-A1, where the first set of probes specific for a SNP in the target sequence contains an endcapped hairpin tunable portion tuned to melt at 60° C. and the second set of probes specific for a different SNP in the target sequence contains an endcapped hairpin tunable portion tuned to melt at 80° C. When both SNPs are present in the target sequence, FRET signals from the FRET pair A1-D1 are detected at both 60° C. and 80° C. Likewise, for two different SNPs present in the target sequence, a first set of probes containing an endcapped hairpin tunable portion are tuned to melt at 65° C. while a second set of probes containing an endcapped hairpin tunable portion are tuned to melt at 85° C. and both sets of probes bear the donor-acceptor FRET pair A2-D2, such that when both SNPs are present, FRET signals from the FRET pair A2-D2 are detected at both 65° C. and 85° C. Similarly, FRET signals for the FRET pair A3-D3 can be detected at 70° C. and 90° C. when two probe sets specific for a third set of SNPs contain endcapped hairpin tunable portions tuned to melt at 70° C. and 90° C. and both SNPs are present in the target sequence. For the FRET pairs A4-D4, A5-D5 and A6-D6, only one signal, arising from probes bearing endcapped hairpin tunable portions tuned to melt at 75° C., 80° C., and 85° C. are shown.

The first and second oligonucleotide probes of a probe set each contain either an acceptor or donor group to facilitate FRET detection. Donor and acceptor fluorophore groups useful for FRET detection are well known in the art. Donor groups include Alexa Fluor 350, Marina Blue, Pacific Orange, Alexa Fluor 405, Pacific Blue, Alexa Fluor 430, Fluorescein and it's derivatives, Alexa Fluor 488, Oregon Green 488, Alexa Fluor 500, Alexa Fluor 514, Oregon Green 514, Alexa Fluor 532, Alexa Fluor 546, Cy3, Alexa Fluor 555, Tetramethylrhodamine and it's derivatives, Alexa Fluor 568, Cy 3.5 Alexa Fluor 594, Texas Red, Alexa Fluor 610, Alexa Fluor 633, Cy 5, Alexa Fluor 647, Alexa Fluor 660, Alexa Fluor 680, Cy 5.5, and Alexa Fluor 700. Likewise, acceptor groups include Marina Blue, Pacific Orange, Alexa Fluor 405, Pacific Blue, Alexa Fluor 430, Fluorescein and it's derivatives, Alexa Fluor 488, Oregon Green 488, Alexa Fluor 500, Alexa Fluor 514, Oregon Green 514, Alexa Fluor 532, Alexa Fluor 546, Cy3, Alexa Fluor 555, Tetramethylrhodamine and it's derivatives, Alexa Fluor 568, Cy 3.5 Alexa Fluor 594, Texas Red, Alexa Fluor 610, Alexa Fluor 633, Cy 5, Alexa Fluor 647, Alexa Fluor 660, Alexa Fluor 680, Cy 5.5, Alexa Fluor 700, and Alexa Fluor 750.

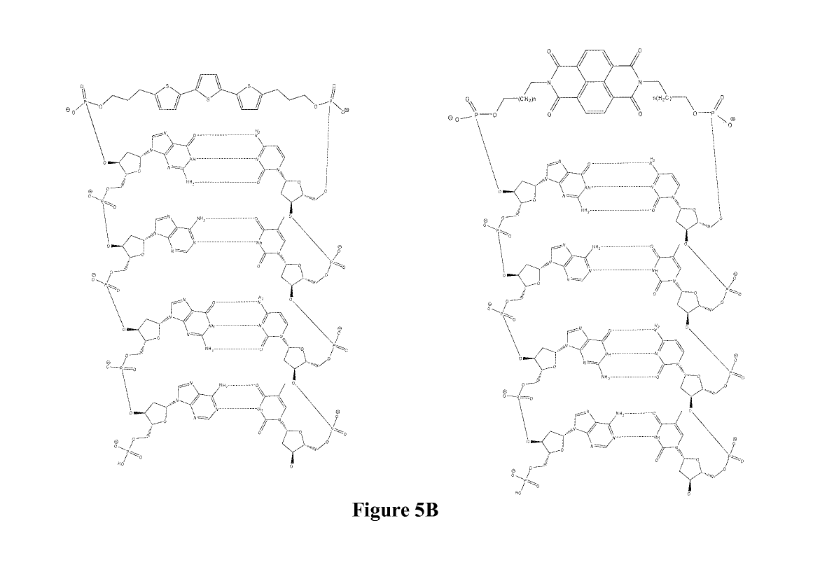

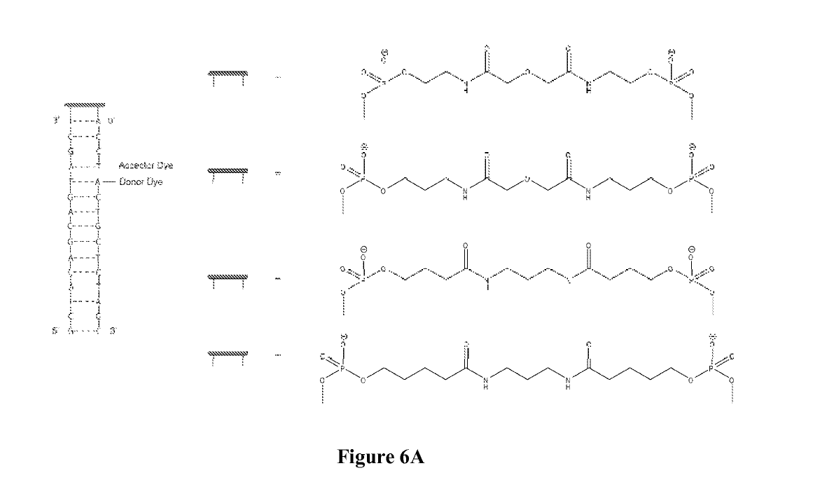

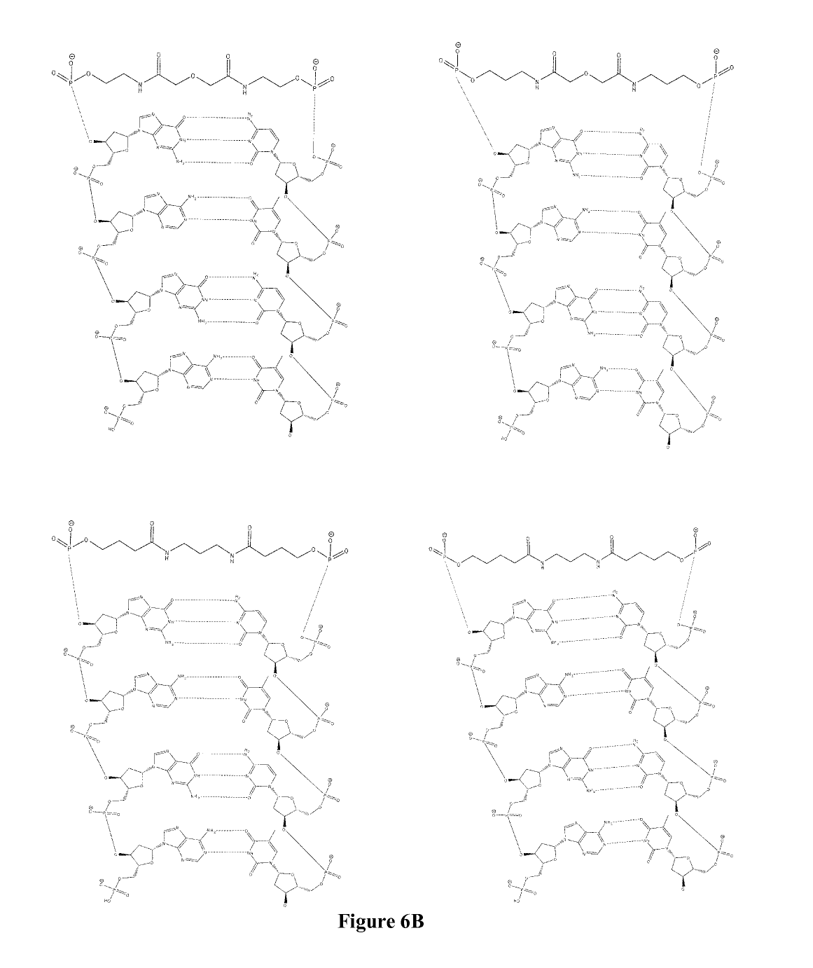

One of the oligonucleotide probes in a probe set of the present invention contains an endcapped hairpin. The endcapped hairpin consists of an endcap molecule and a short complementary oligonucleotide duplex (See FIGS. 5A and 6A). The oligonucleotides of the complementary oligonucleotide duplex can range between three and ten nucleotides in length. More preferably however, the oligonucleotides of the duplex are between four and six nucleotides in length.

Endcap molecules are organic molecules that covalently link the 5′-terminus of an oligonucleotide in the oligonucleotide duplex with the 3′-terminus of its complementary oligonucleotide. Endcaps are synthesized as phosphoramidites and are incorporated into the oligonucleotide probes using standard reagents during automated synthesis as previously described (Pingle et al., “Synthesis of Endcap Dimethoxytrityl Phosphoramidites for Endcapped Oligonucleotides,” Curr. Prot. Nucl. Acids. Chem. pp. 5.6.1-5.6.15 (2002) and Ng et al., “Endcaps for Stabilizing Short DNA Duplexes,” Nucleosides Nucleotides and Nucl. Acids 22(5-8):1635-1637 (2003), which are hereby incorporated by reference in their entirety).

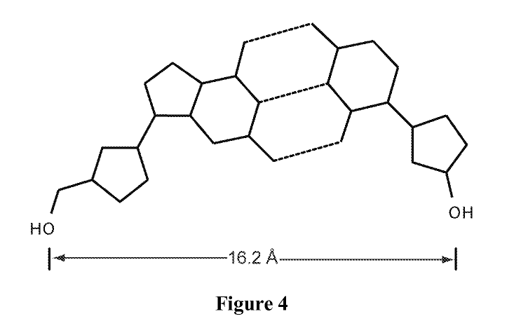

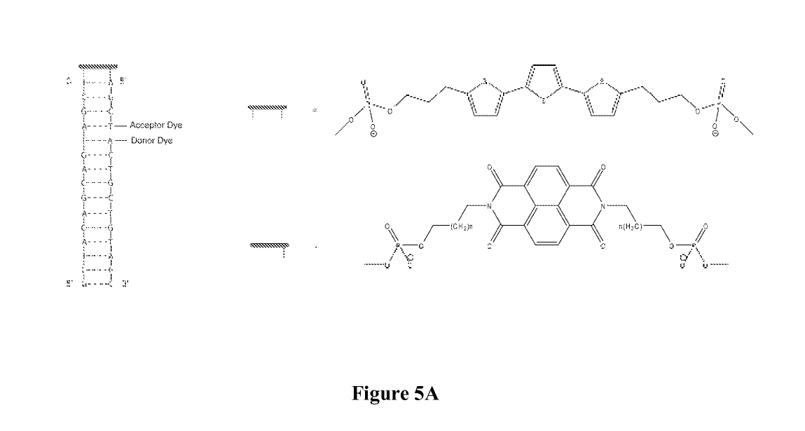

Endcaps are designed to closely mimic the β-DNA interstrand distance. The β-DNA intrastrand distance can vary slightly depending on the specific nucleotide base pair present, but typically averages 16.2 Å (See FIG. 4). The endcaps are designed to increase the stability of the oligonucleotide duplex through base stacking interactions. Endcaps do not perturb the structure of the DNA helix and are stable to nucleases. A particular endcap-oligonucleotide duplex can be designed or “tuned” such that the duplex will melt at a specific temperature. The endcap molecule modulates the melting such that the melting transition occurs over a narrow temperature range. Endcaps may be aromatic or aliphatic.

Aromatic endcaps provide a higher degree of stabilization compared to aliphatic endcaps. The aromatic endcaps shown in FIG. 5A can stabilize 4 bp stems with 2 AT and 2 GC base pairs to have Tm values ranging from 53° C. to 75° C., depending on the endcap and the sequence of the stem. Attachment of the aromatic endcap molecules to the 3′ and 5′ termini of a pair of complementary oligonucleotides is shown in FIG. 5B. A few examples of aliphatic endcaps are shown in FIG. 6A. These endcaps are more hydrophilic and can stabilize 4 bp stems to have Tm values between 39° C. and 56° C. For comparison, the same 4 bp stems joined by a tetranucleotide “T” loop or “A” loop (natural hairpins) result in Tm values between 39 to 62° C. Attachment of the aliphatic endcap molecules to the 3′ and 5′ termini of a pair of complementary oligonucleotides is shown in FIG. 6B.

The hybridization step of the ligase detection reaction, which is preferably a thermal hybridization treatment, discriminates between nucleotide sequences based on a distinguishing nucleotide at the ligation junctions. The difference between the target nucleotide sequences can be, for example, a single nucleic acid base difference, a nucleic acid deletion, a nucleic acid insertion, or rearrangement. Such sequence differences involving more than one base can also be detected. Preferably, the oligonucleotide probe sets are substantially the same length so that they hybridize to target nucleotide sequences at substantially similar hybridization conditions. As a result, the process of the present invention is able to detect infectious diseases and is useful in environmental monitoring, forensics, and food science.

A wide variety of infectious diseases can be detected by the process of the present invention. Typically, these are caused by bacterial, viral, parasite, and fungal infectious agents, The resistance of various infectious agents to drugs can also be determined using the present invention.

Bacterial infectious agents which can be detected by the present invention include Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Acitnomycetes.

Fungal infectious agents which can be detected by the present invention include Cryptococcus neoformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis.

Viral infectious agents which can be detected by the present invention include human immunodeficiency virus, human T-cell lymphocytotrophic virus, hepatitis viruses (e.g., Hepatitis B Virus and Hepatitis C Virus), Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

Parasitic agents which can be detected by the present invention include Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

The present invention is also useful for detection of drug resistance by infectious agents. For example, vancomycin-resistant Enterococcus faecium, methicillin-resistant Staphylococcus aureus, penicillin-resistant Streptococcus pneumoniae, multi-drug resistant Mycobacterium tuberculosis, and AZT-resistant human immunodeficiency virus can all be identified with the present invention.

In one embodiment of the present invention, the target sequence is preferably amplified by an initial target nucleic acid amplification procedure. This increases the quantity of the target nucleotide sequence in the sample. The initial target nucleic acid amplification may be accomplished using the polymerase chain reaction process as fully described in Erlich et al., “Recent Advances in the Polymerase Chain Reaction,” Science 252: 1643-50 (1991); Innis et al., PCR Protocols: A Guide to Methods and Applications, Academic Press: New York (1990); and Saiki et al., “Primer-directed Enzymatic Amplification of DNA with a Thermostable DNA Polymerase,” Science 239: 487-91 (1988), which are hereby incorporated by reference in their entirety. In this embodiment, a plurality of oligonucleotide primer sets and a DNA polymerase are provided and blended with the sample to form a polymerase chain reaction mixture. The polymerase chain reaction mixture is subjected to one or more polymerase chain reaction cycles comprising a denaturation treatment, wherein hybridized nucleic acid sequences are separated, a hybridization treatment, wherein the oligonucleotide primers hybridize to their complementary target-specific portions, and an extension treatment, wherein the hybridized oligonucleotide primers are extended to form extension products. Following the polymerase chain reaction cycles, the ligase detection reaction mixture is formed by blending the extension products of the polymerase reaction mixture rather than the sample with the ligase and the plurality of oligonucleotide probes.

Although the polymerase chain reaction process is a preferred amplification procedure, self-sustained sequence replication as taught by Guatelli et al., “Isothermal, in vitro Amplification of Nucleic Acids by a Multienzyme Reaction Modeled After Retroviral Replication,” Proc. Natl. Acad. Sci. USA 87: 1874-78 (1990), which is hereby incorporated by reference in its entirety, or Q-β replicase-mediated RNA amplification disclosed in Kramer et al., “Replicatable RNA Reporters,” Nature 339: 401-02 (1989), which is hereby incorporated by reference in its entirety, can also be used to amplify the target nucleic acid prior to the ligase detection reaction.

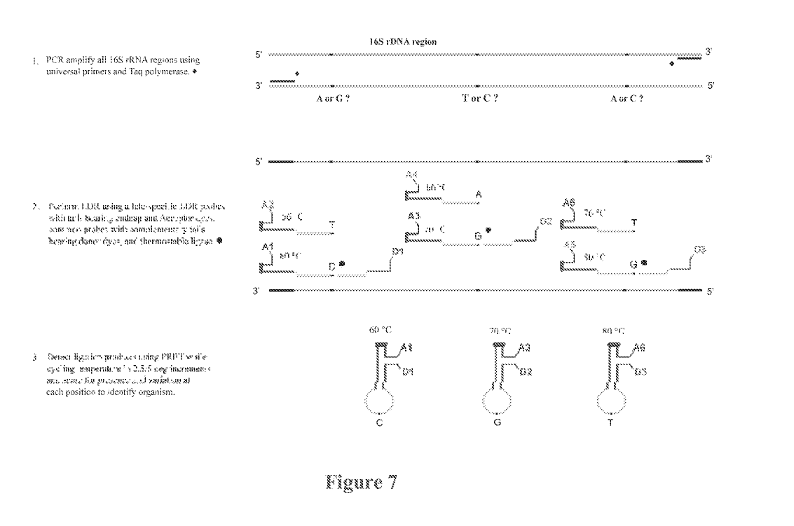

FIG. 7 is a schematic representation of the detection and identification of bacterial pathogens using the 16s ribosomal RNA bacterial gene in accordance with the methods of the present invention. In this embodiment, oligonucleotide primers are designed to PCR amplify the target sequence as shown in step 1 of FIG. 7. After PCR amplification, the polymerase is inactivated. The oligonucleotide probes are designed to identify different SNPs within target genes, which in turn can be used to distinguish different strains or serotypes of pathogens. Up to twelve simultaneous signals may be read, and the distribution of simultaneous signals is chosen such that there is substantial difference in the Tm values of the tunable probes of the two potential ligation products.

As shown in step 2 of FIG. 7, one probe group consists of a first oligonucleotide probe having a 3′ target specific portion directed to one allele and a 5′ portion containing a tunable portion with an end-capped hairpin, which is tuned to have a Tm value of 60° C. with an acceptor group, A1. The probe group further consists of a second first oligonucleotide probe having a 3′ target specific portion directed to a second allele and a 5′ portion containing a tunable portion with an end-capped hairpin, which is tuned to have a Tm value of 56° C. with an acceptor group, A2. In this example, acceptor groups A1 and A2 can be the same or different fluorophores. The corresponding downstream second oligonucleotide probe has a 5′ target specific portion and 3′ tunable panhandle probe portion with a donor group. When there is perfect complementarity at the junction between one of the first probes and the corresponding second downstream probe and the target, the two probes are ligated together. As shown in step 3 of FIG. 7, upon denaturing the ligation product from the target, the downstream tunable probe portion forms a double-stranded region to the tunable probe portion of the upstream probe at the Tm of the tunable regions, whereupon the donor and acceptor groups of the upstream and downstream probes are in close enough proximity allowing for a FRET to occur.

As further shown in step 2 of FIG. 7, a similar design is used for other probe groups, each probe group directed to a different nucleic acid target and having tunable probe portions with differing melting temperatures. The second probe group in step 2 of FIG. 7 has first oligonucleotide probes having tunable probe portions tuned to have Tm values of 66° C. or 70° C., respectively, and a third probe group has first oligonucleotide probes having tunable probe portions tuned to have Tm values of 76° C. and 80° C., respectively. When multiple products are produced (either within a probe group or between probe groups) they are denatured from the target sequence and then slowly cooled to renature, forming the internally hybridized products. Under these conditions, the product with the higher Tm (i.e. 80° C.) forms first, bringing the donor and acceptor molecules in close proximity resulting in the generation and detection of the FRET signal. FRET signal increases rapidly as the first internally hybridized product forms, with a Tm at 80° C. As the temperature is lowered further, FRET signal rises again with the formation of the internally hybridized ligation products having a Tm of 70° C. and then those products have a Tm of 60° C.

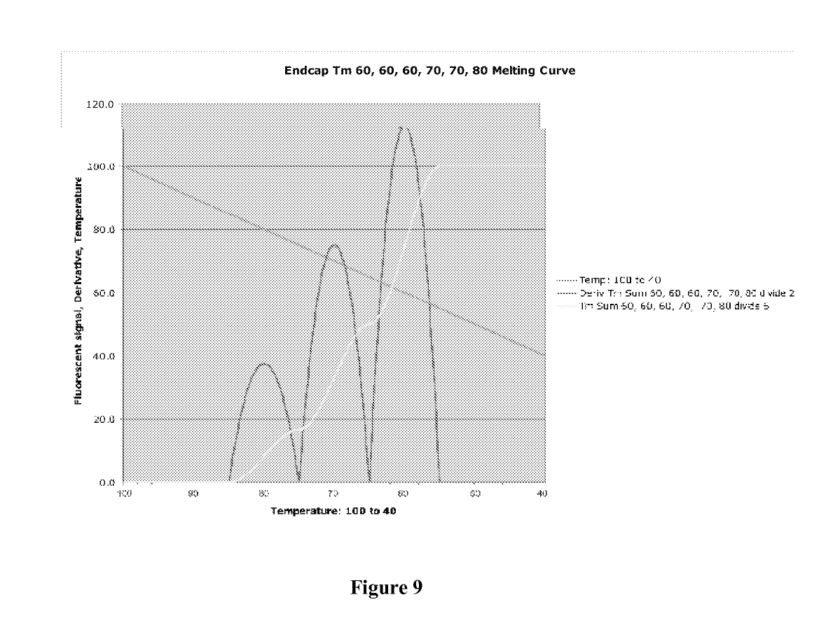

FIGS. 9 and 10 show representative melting curves for single color oligonucleotide probe FRET readout where three distinct ligation products are formed as illustrated in FIG. 7. In these examples, the products formed have Tm values that are 10° C. apart. After the ligation detection reaction, the products are denatured from the template and slowly cooled. The yellow curve in both figures displays the melting curve which tracks intensity of the FRET signal generated by the formation of the internally hybridized ligation products with the decrease in temperature. The red curve represents the derivative of the melting curve. The melting peaks of the derivative curves represent the melting temperature of the tunable and endapped portions of an internally hybridized ligation product.

Varying amounts of ligation products are formed in the ligation detection reaction depending on the amount of target sequence and the efficiency of the ligation at that locus. The height of the melting peaks of the derivative curves generated in the melt curve analysis correspond to the relative amounts of target sequence detected. As shown in FIG. 9, there is three-fold more LDR product having a Tm of 60° C. than LDR product having a Tm of 80° C. Likewise, there is two-fold more LDR product having a Tm of 70° C. than LDR product having a Tm of 80° C. FIG. 10 demonstrates the opposite, where the amount of LDR product having a Tm of 80° C. exceeds the LDR products having a Tm of 70° C. and 60° C.

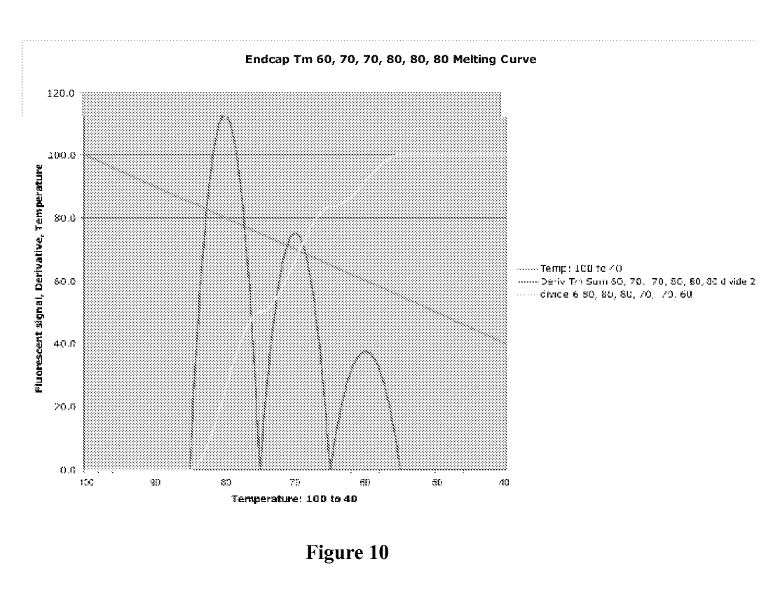

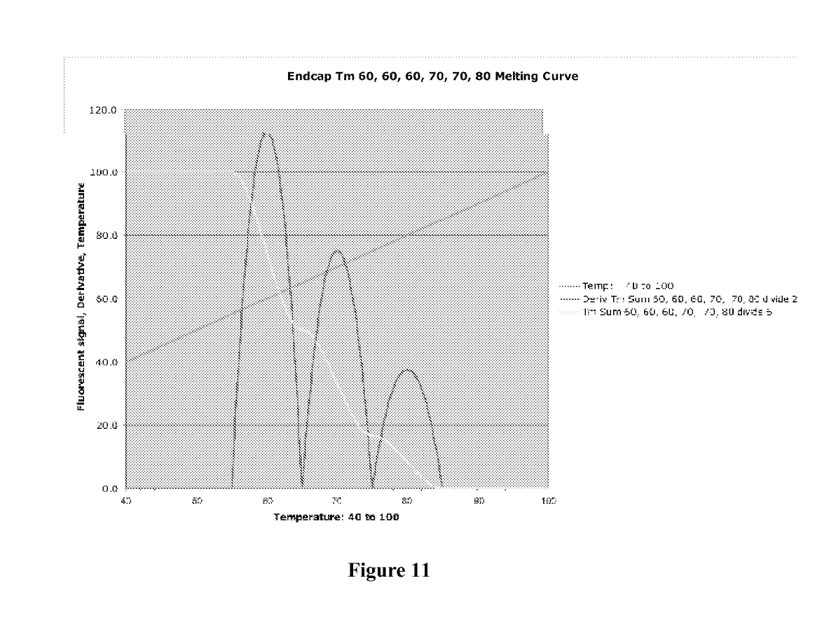

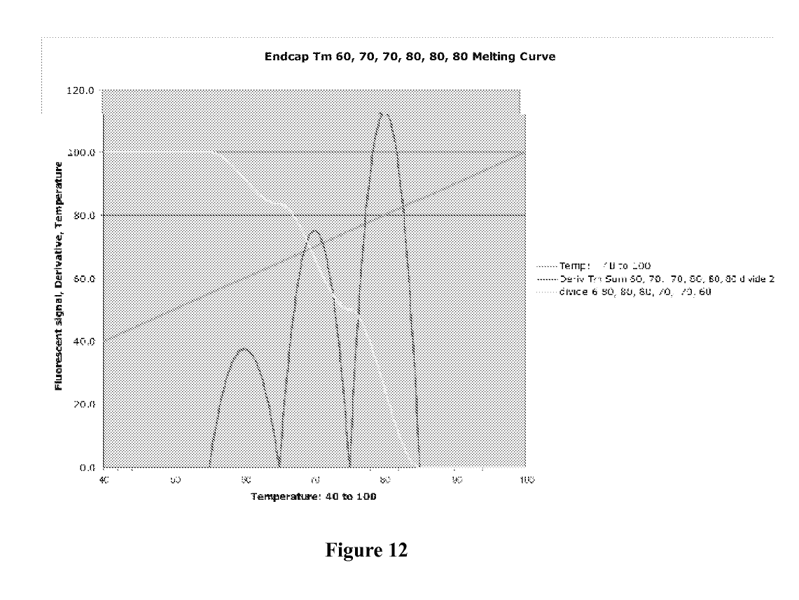

FIGS. 11 and 12 also show representative melting curves for single color oligonucleotide probe FRET readout where three distinct ligation products are formed as illustrated in FIG. 7. In these examples, the ligation products are denatured from the template and the sample is cooled to 40° C., allowing the internally hybridized products to form at their designated melting temperatures. The temperature is then slowly raised from 40° C. to 100° C., allowing the internally hybridized products to denature or melt at their designated melting temperatures. A melting curve (yellow curve) is generated by detecting the change in FRET signal, generated by the internally hybridized products, with the increasing temperature. The red curve represents the derivative of the melting curve, where each melting peak of the derivative curve represents the melting temperature of the tunable and endcapped portions of a specific internally hybridized ligation product.

Using this approach, i.e. detecting FRET over an increase in the temperature from 40° C.-100° C., the relative amounts of target sequence present in the sample can be distinguished. As shown in FIG. 11, there is three-fold more LDR product having a Tm of 60° C. than LDR product having a Tm of 80° C. Likewise, there is two-fold more LDR product having a Tm of 70° C. than LDR product having a Tm of 80° C., FIG. 12 demonstrates the opposite, where the amount of LDR product having a Tm of 80° C. exceeds the LDR products having a Tm of 70° C. and 60° C.

The probes can be designed in a hierarchical way such that product from the lower tunable Tm generally indicates the presence of a broader category of target (e.g. pathogen) than the more specific species identification provided by the higher probe. Further, the probes may be added in quantities that assure the lower Tm tunable product is produced in amounts equal to or greater than the amount of product for the higher tunable ligation product. The total amount of ligation product formed is not as important as getting a clean melt curve to accurately identify the Tm of a given ligation product. The advantage of using tunable probes is that the ligation product FRET signal is based on the tunable portion of the probes, and not on the Tm of the probes hybridizing to the target. Thus, this approach can tolerate sequence variation in the target and provides considerable advantage in correctly identifying the target(s) compared with other multiplexed approaches.

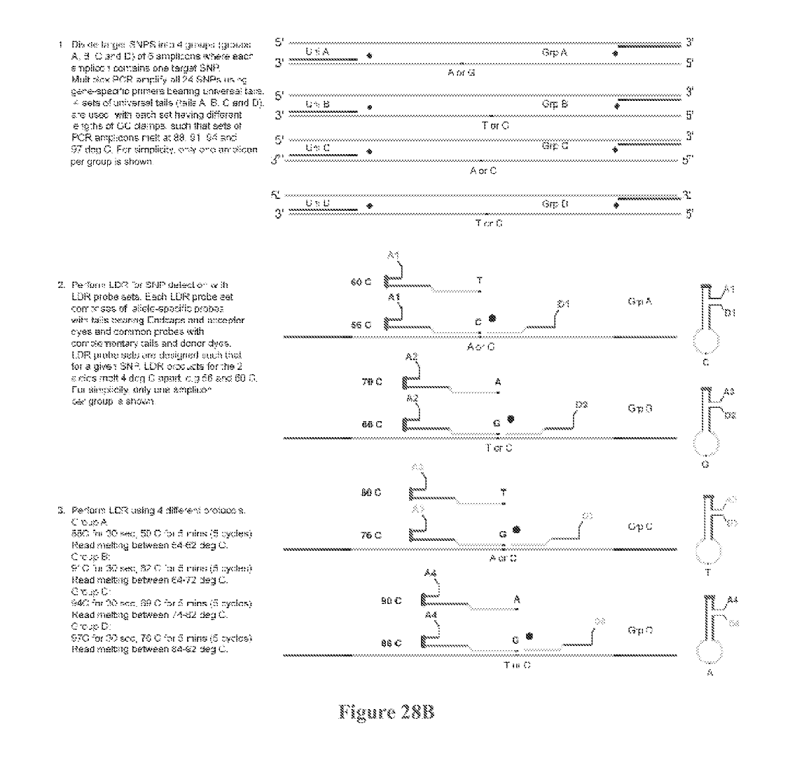

In a variation of the method shown in FIG. 7, up to forty-eight simultaneous signals from up to forty-eight different genotypes can be readily determined. In this embodiment, the first PCR phase of the method comprises a multiplex amplification of the target DNA. Oligonucleotide primers are designed to amplify multiple regions of multiple targets using the PCR. Target genes can be divided into groups (e.g. four groups A-D), with each group containing between one and six amplicons. The number of amplicons per group is limited only by the number of FRET acceptor-dye pairs. Each amplicon contains a SNP to be investigated. In one embodiment the oligonucleotide primers are designed so that all target gene extension products will melt at the same time (see step 1 of FIG. 28A).

In accordance with this embodiment of the present invention, and as shown in FIG. 28A, the oligonucleotide primers preferably comprise a target-specific portion and an universal tail portion. The extension product sequences that are formed as a result of the polymerase chain reaction cycles comprise a 5′ universal tail portion, a target portion, and a 3′ universal tail portion.

The initial amplification of all target genes (Groups A-D) is done under conditions that push all the amplifications to completion, (e.g. cycles of 97° C. (30 seconds, for denaturation); 65° C. (1 minute for primer binding and initial extension); 75° C. (2 minutes to complete primer extension)). After PCR, the polymerase is inactivated and the ligase detection reaction mixture is formed.

As described supra, the oligonucleotide probes of the present invention are capable of distinguishing multiple allele differences at one or more nucleotide positions in a single or multiple target nucleic acid molecule. The oligonucleotide probes can be designed to identify allele difference (i.e. SNPs) within targets for each group of extension products in a sequential order. For example, the target-specific portions of the oligonucleotide probes can be designed to hybridize and ligate to the target sequence at particular temperatures (e.g. 55° C., 62° C., 69° C., and 76° C.).

After the ligase detection reaction phase of the method is completed, the FRET signals are detected by performing a melt curve analysis. The same FRET signals from ligation products within the same probe group are distinguished by the melting peaks of a first derivative of the melt curve, which differ by at least 4° C. FRET signals from ligation products of a first probe group are distinguished from the same FRET signals from ligation products of a second probe group, by the melting peaks of a first derivative of the melt curve, which are at least 6° C. higher for the second group than for the first group.

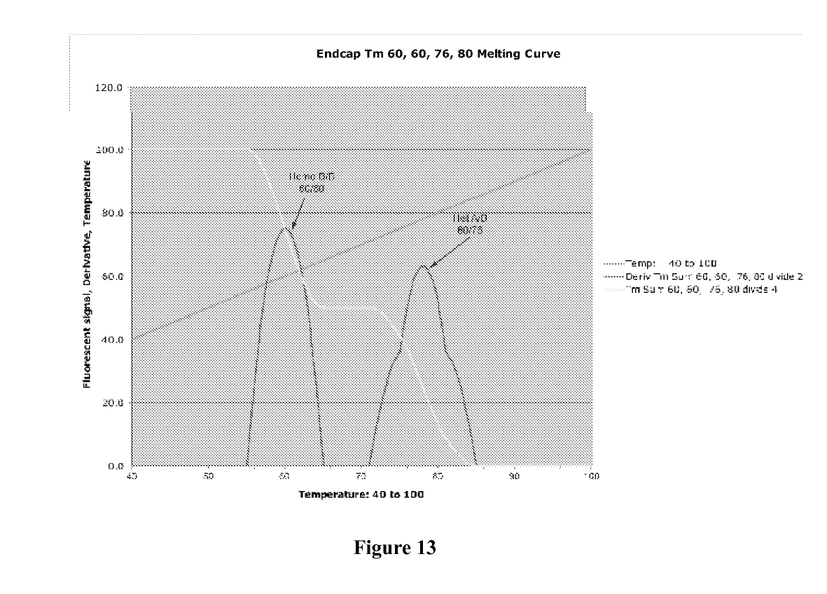

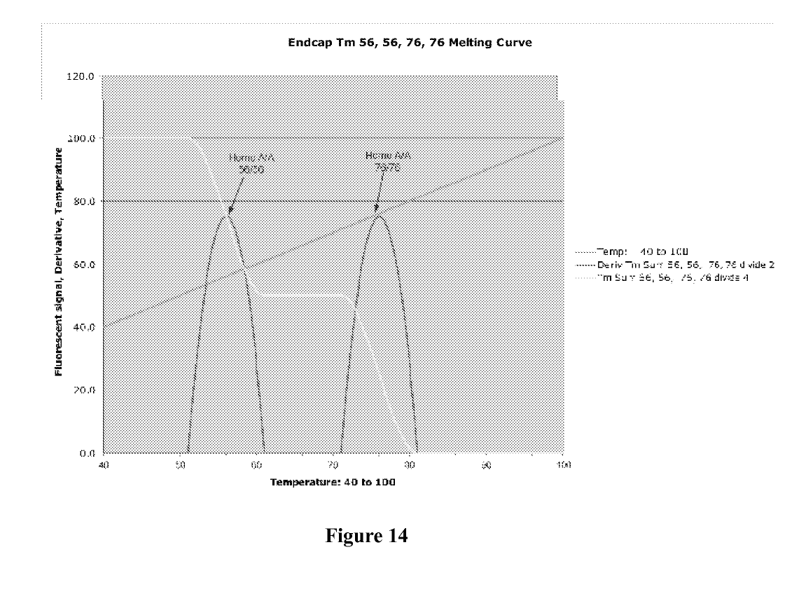

FIG. 13 illustrates a typical melt curve resulting from the use of a single donor-acceptor FRET pair to investigate only two SNPs, each SNP belonging to a different amplicon (i.e. different target regions amplified by the oligonucleotide primers). The yellow curve represents the melting curve and the red curve represents the derivative of the melting curve. The light blue line shows the temperature ramp from 40° C.-100° C. Note the difference in shape of the derivative curve for the homozygous SNP that produces a single melting peak at 60° C. versus the heterozygous SNP that produces overlapping melting peaks at 76 and 80° C. FIG. 14 shows a typical melting curve where two different homozygous SNPs are detected.

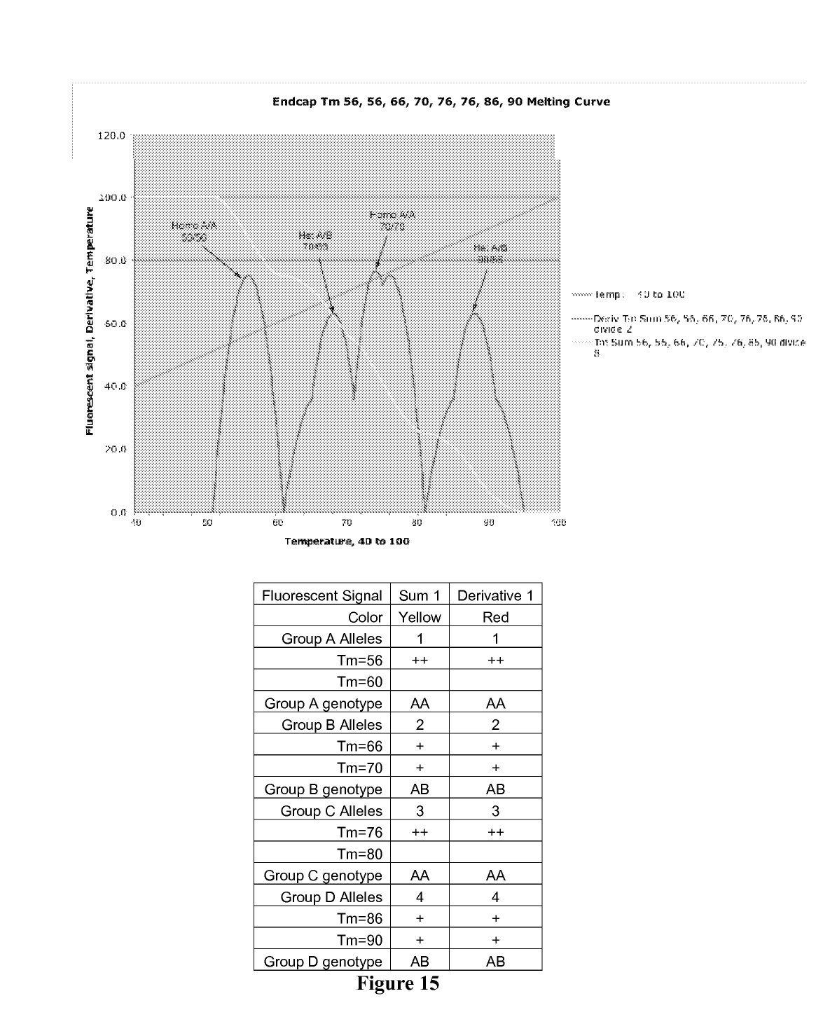

Four different SNPs (one belonging to each of the four groups A-D) can also be detected using a single donor-acceptor FRET pair. The presence of one or both alleles at each SNP is distinguished by looking at the melting peaks of the derivative curve (red curve) (See FIGS. 15-18). In these figures, the Group A SNPs are read between 54-62° C., Group B between 64-72° C., Group C between 74-82° C., and Group D between 84-92° C.

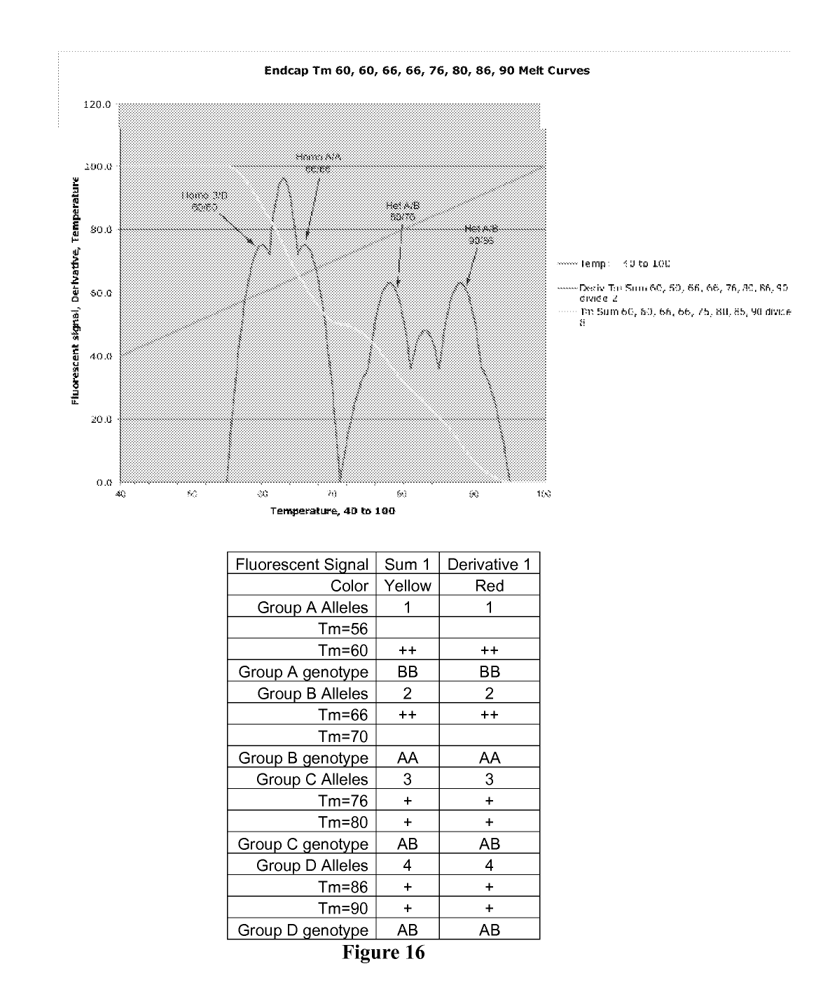

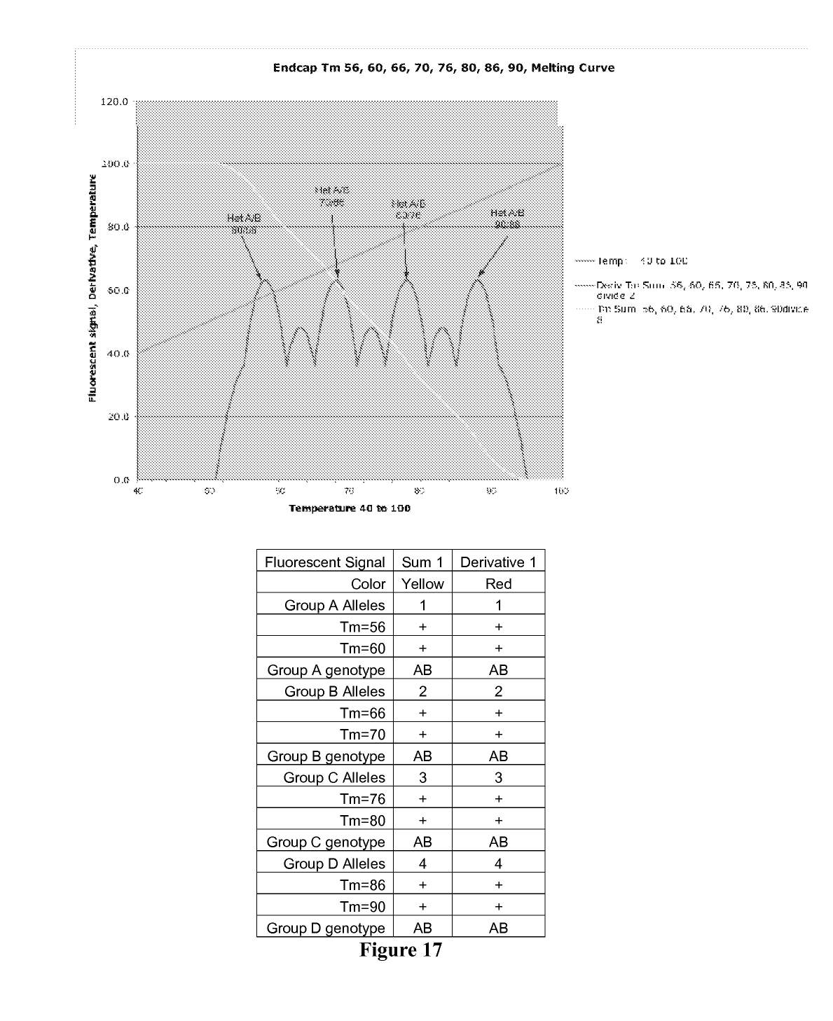

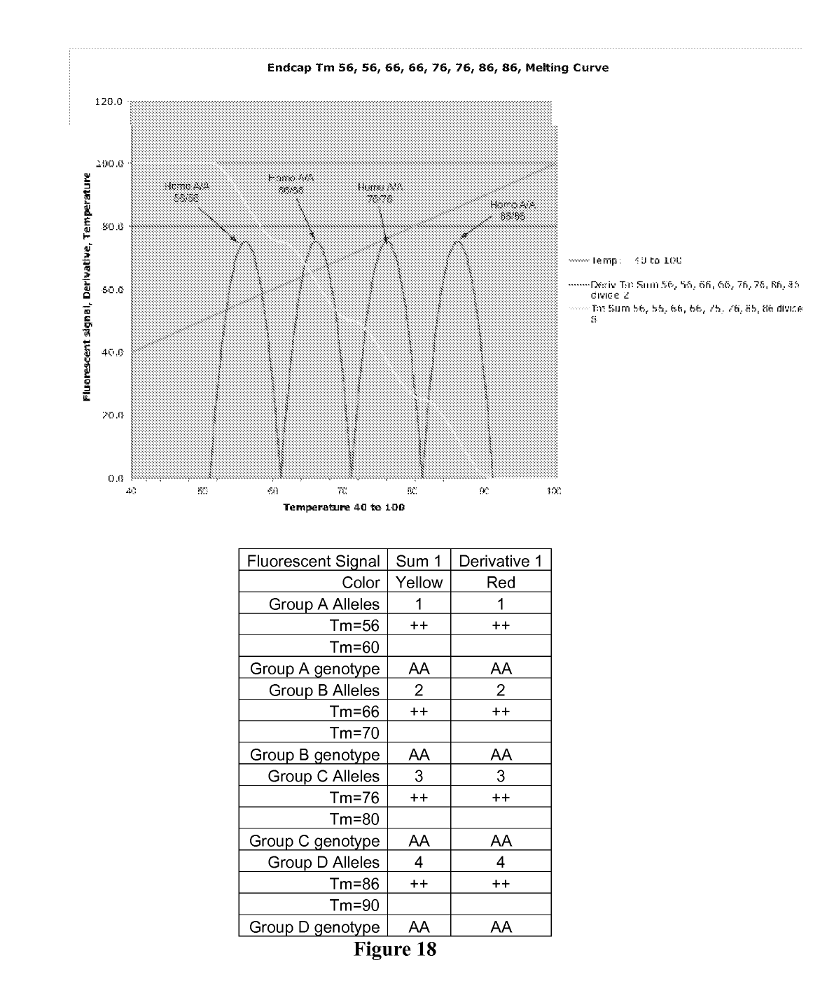

In the melt curve of FIG. 15, Group A and C SNPs are homozygous for allele A, and Group B and D SNPs are heterozygous for both A and B alleles. In the melt curve of FIG. 16, Group A SNPs are homozygous for the B allele, Group B SNPs are homozygous for the A allele, and Groups C and D are heterozygous for both A and B alleles. In the melt curve of FIG. 17, all Groups A-D are heterozygous for both the A and B alleles. In the melt curve of FIG. 18, all Groups A-D are homozygous for the A allele.

FRET signals can also be measured by denaturing all the ligation products at 100° C. and slowly cooling the sample to down to 40° C. Over the decrease in temperature, the tunable portions of the ligation products anneal, forming the internally hybridized ligation probes and the FRET is detected. In this variation, FRET from the higher melting probes is detected first, followed by the detection of FRET from the lower melting probes.

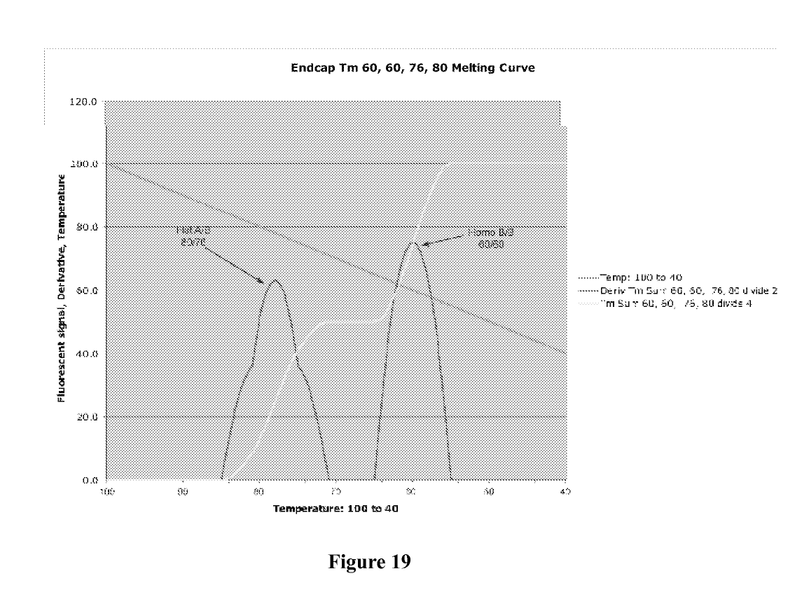

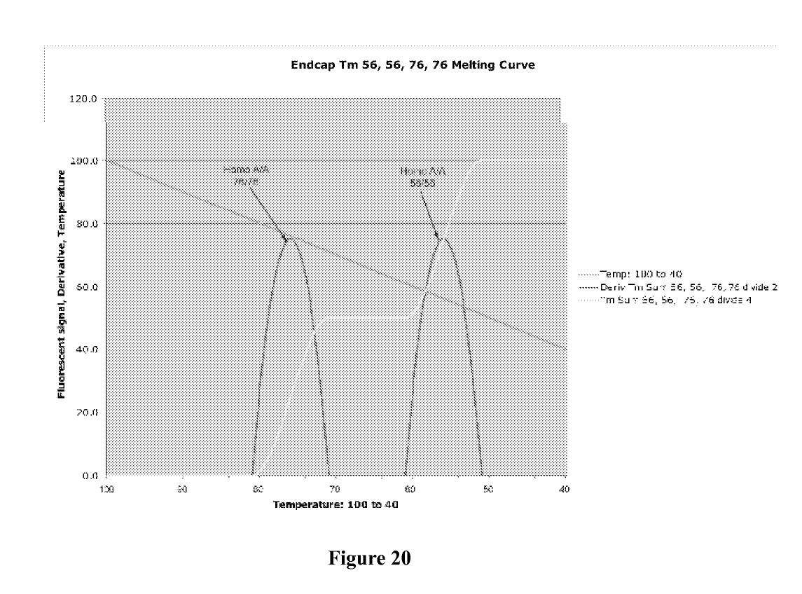

FIGS. 19 and 20 show how this variation will alter the shape of the melt curve when two SNPs are detected. In FIG. 19, SNP1 is homozygous for allele B, producing a peak at 60° C. In contrast, SNP2 is heterozygous for both A and B alleles, producing a combination of two separate peaks, one for allele A at 76° C. and the other for allele B at 80° C. In FIG. 20, both SNPs are homozygous for allele A.

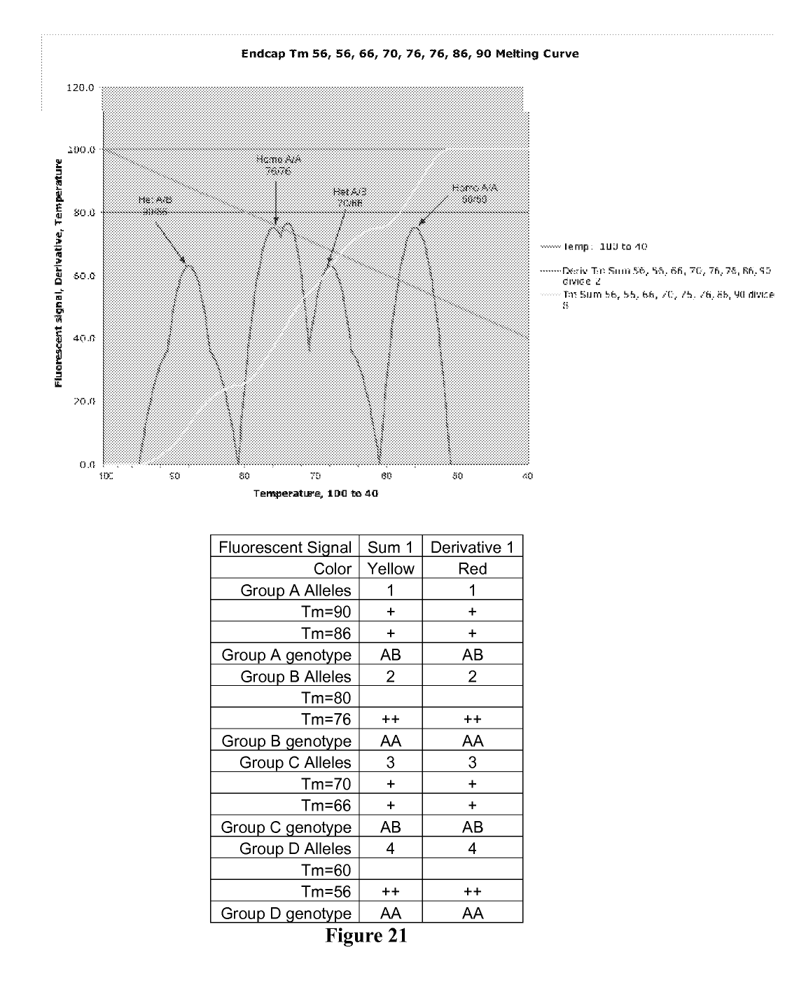

FIGS. 21-24 show the detection of four different SNPs (one belonging to each of the four Groups A-D) using a single donor-acceptor FRET pair when the temperature is slowly cooled from 100° C. to 40° C.

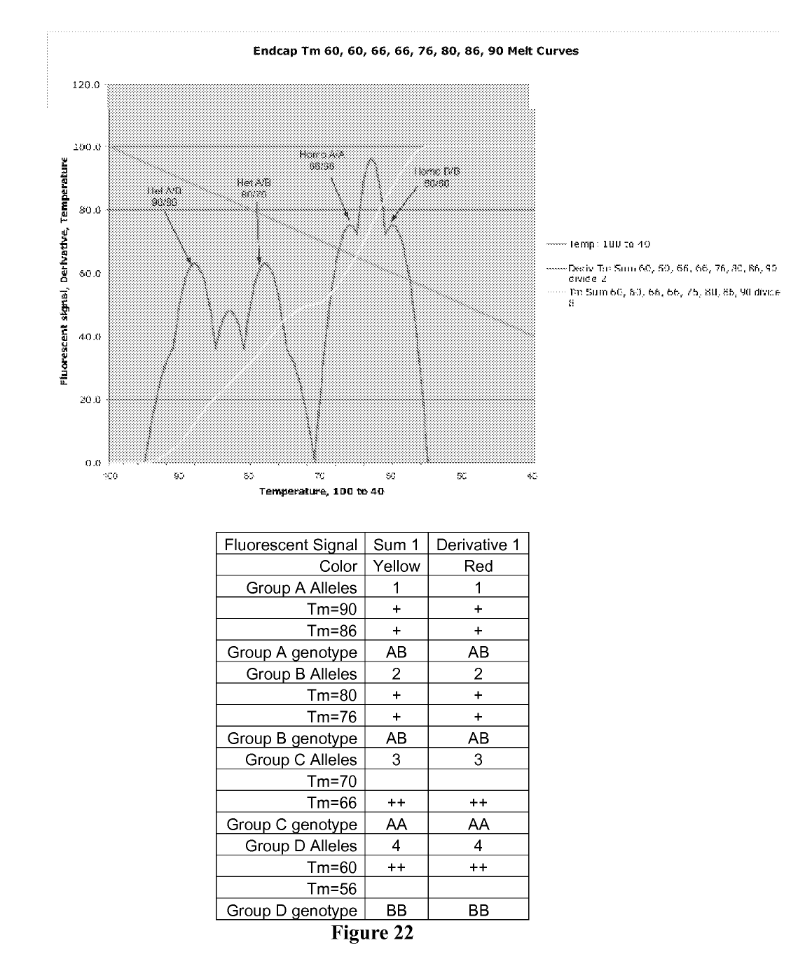

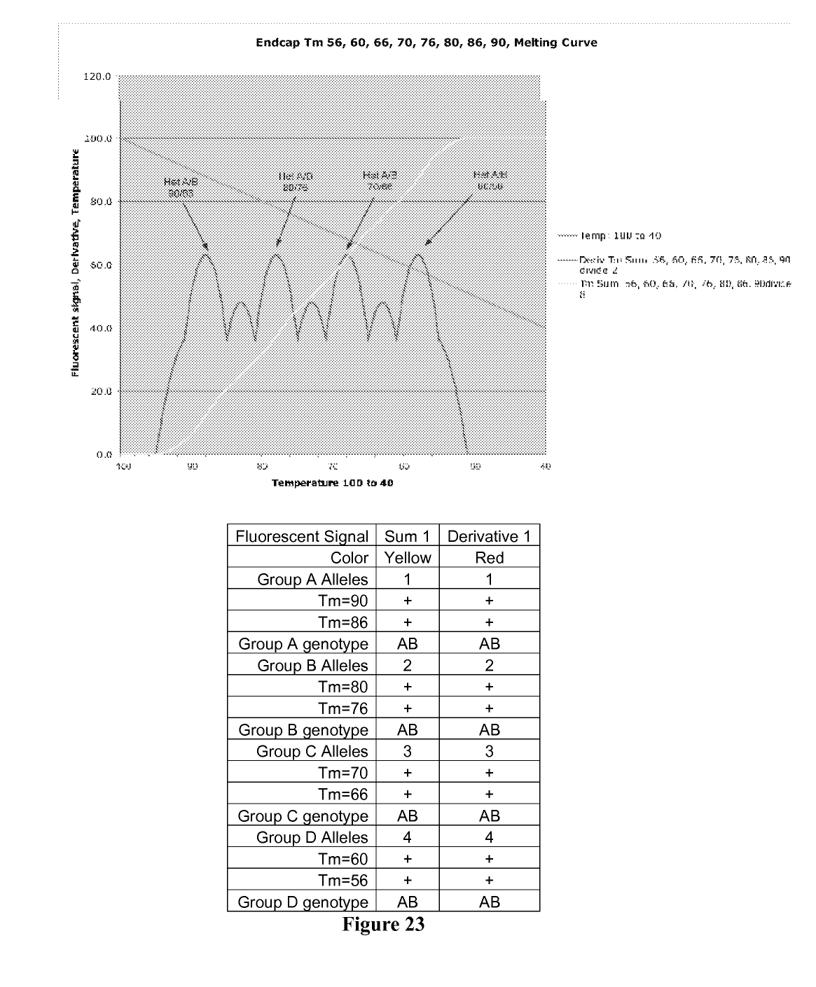

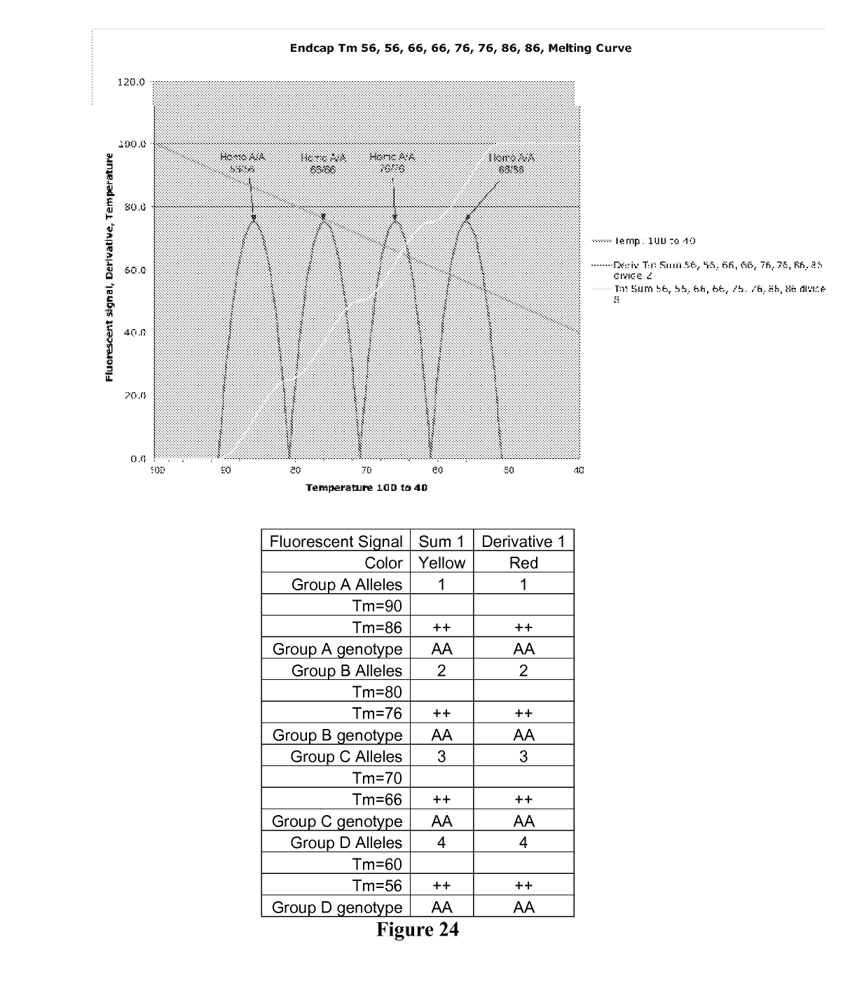

In the melt curve of FIG. 21, Groups A and C are heterozygous for both the A and B alleles and Groups B and D are homozygous for the A allele. In the melt curve of FIG. 22, Groups A and B are heterozygous for the A and B allele, Group C is homozygous for the A allele and Group D is homozygous for the B allele. In the melt curve of FIG. 23, all Groups A-D are heterozygous for the A and B allele. In the melt curve of FIG. 24, all Groups A-D are homozygous for the A allele.

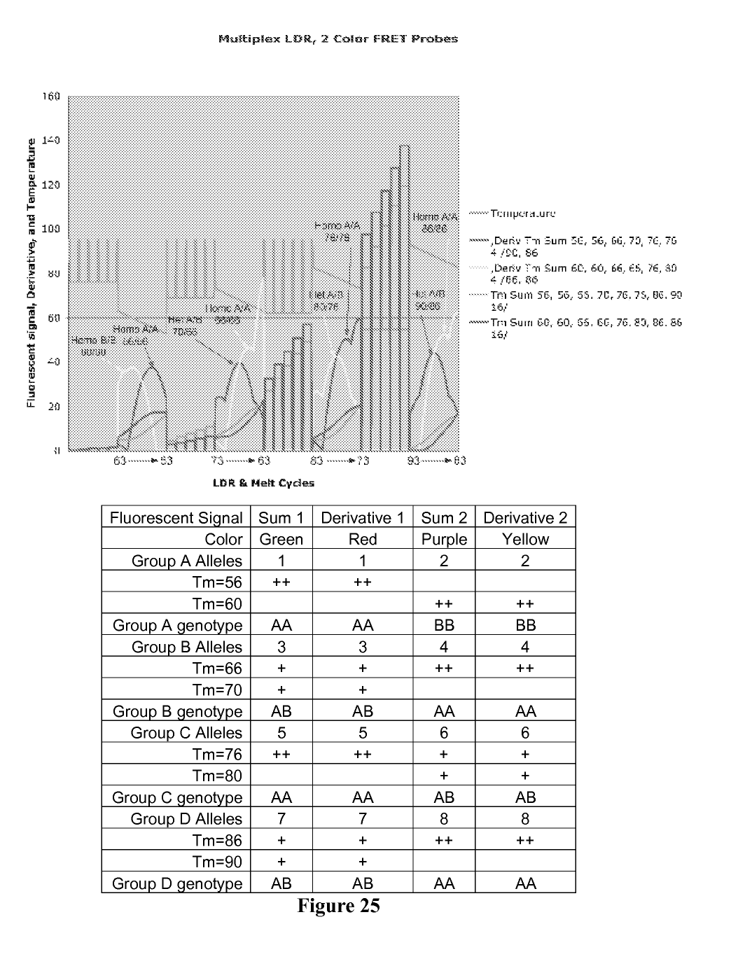

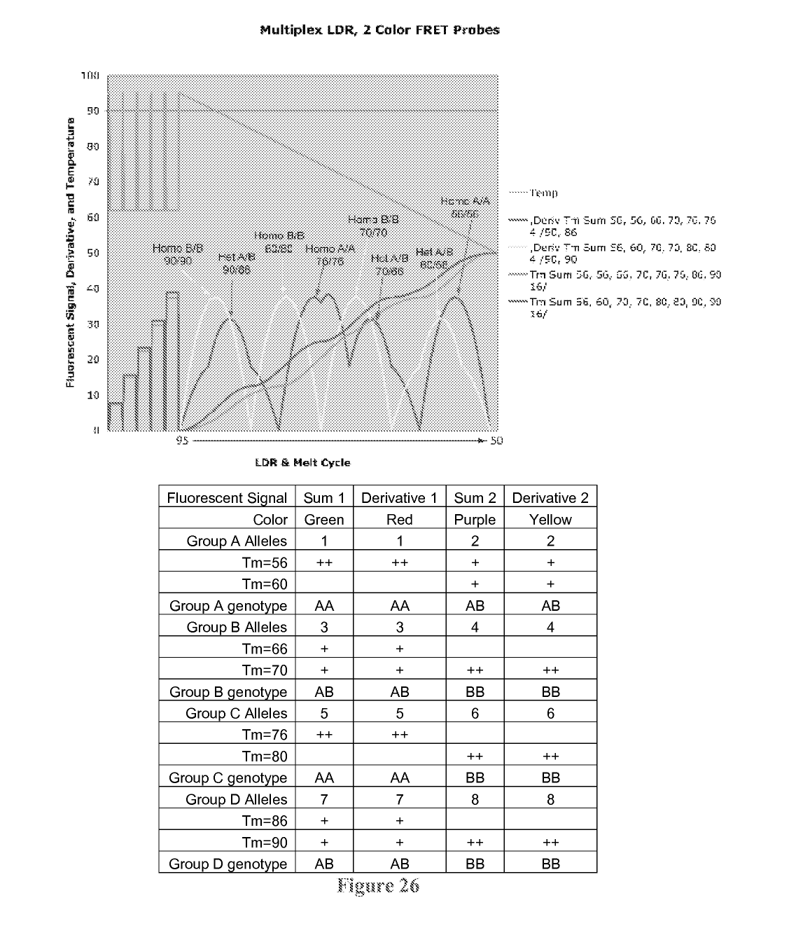

The above examples used a single FRET donor-acceptor pair to identify one SNP per group of PCR amplicons. To achieve a higher degree of multiplexing, additional oligonucleotide probes having a distinct donor-acceptor FRET pair directed to a second set of SNPs in each group of PCR amplicons can be utilized. Following five cycles of the ligase detection reaction, the reaction is heated to 100° C. to completely denature the ligation products from the template and slowly cooled to measure the fluorescence from both FRET donor-acceptor pairs. Examples of typical melting curves using oligonucleotide probes in two different colors are shown in FIGS. 25 and 26. In each of these figures, the light blue line tracks the change in temperature during the ligation reaction cycling and FRET detection. The purple and green lines represent the melt curves for the two FRET donor-acceptor pairs, showing the increase in fluorescence signal with change in temperature. The red line represents the derivative of the green melt curve while the yellow line represents the derivative of the purple melt curve.

FIG. 25 shows an embodiment in which the oligonucleotide probes are designed to identify allele differences (i.e. SNPs) within targets for each group of extension products in a sequential order. In this embodiment the target-specific portions of the oligonucleotide probes are designed to hybridize and ligate to the target sequence at particular temperatures (e.g., 55° C., 62° C., 69° C., and 76° C.). FIG. 25 shows the melt curves and their derivative curves for ligation products formed in four separate ligation reactions, performed consecutively. The ligation reaction for Group A targets is performed and the products detected between 63° C.-53° C. The first Group A target is homozygous for the A allele as shown by the melting peak of the red derivative curve, while the second group A target is homozygous for the B allele as shown by the melting peak of the yellow derivative curve. The ligation reaction for Group B targets is performed and the products detected between 73° C.-63° C. The first Group B target is heterozygous for the A and B allele as indicated by the melting peaks of the red derivative curve, while the second Group B target is homozygous for the A allele as indicated by the melting peaks of the yellow derivative curve. The ligation reaction for Group C targets is performed and the products detected between 83° C.-73° C. The first Group C target is homozygous for the A allele as indicated by the melting peaks of the red derivative curve, while the second Group C target is heterozygous for the A and B allele as indicated by the melting peaks of the yellow derivative curve. The ligation reaction for Group D targets is performed and the products detected between 93° C.-83° C. The first Group D target heterozygous for the A and B allele as indicated by the melting peaks of the red derivative curve, while the second Group D target is homozygous for the A allele as indicated by the melting peaks of the yellow derivative curve.

FIG. 26 shows an embodiment in which one ligation reaction (5 cycles) is performed, the ligation reaction sample is heated to 100° C. to denature the ligation products from their target, and a melt curve analysis is performed to determine the genotypes of Group A-D targets. The red curve is the derivative of the green melt curve and the yellow curve is the derivative of the purple melt curve. The green and purple melt curves show the accumulation of FRET signal over the temperature decrease from 100° C.-40° C. The Group A target products are detected at 60° C. and 55° C., Group B target products are detected at 70° C. and 66° C., Group C target products are detected at 80° C. and 77° C., and the Group D target products are detected at 90° C. and 86° C. The genotypes of Group A-D targets are summarized in the table accompanying the melt curve analysis of FIG. 26.

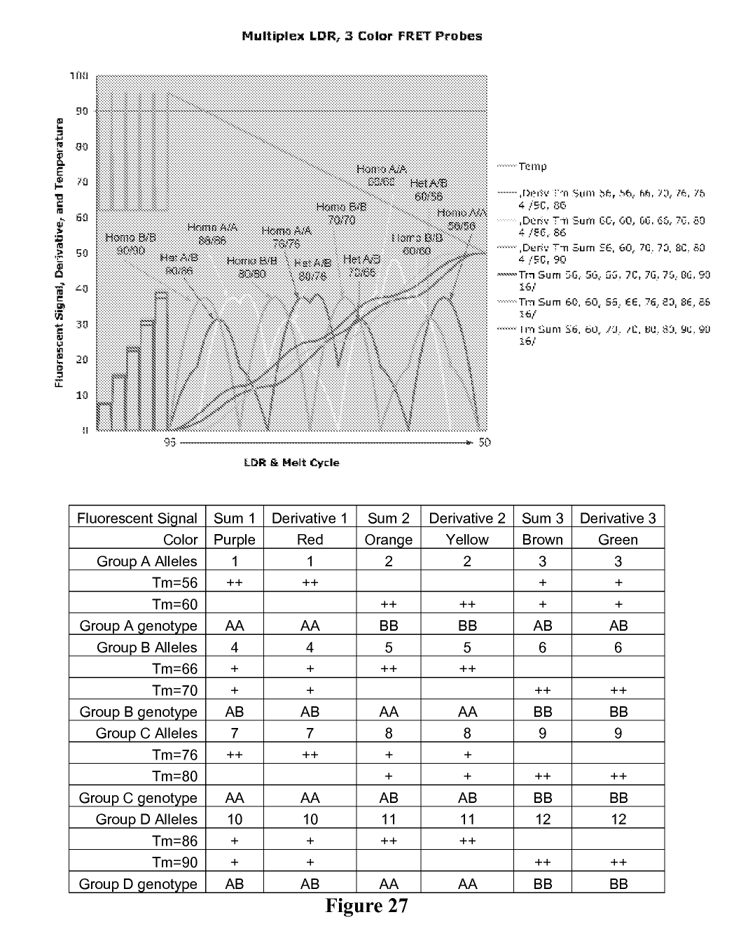

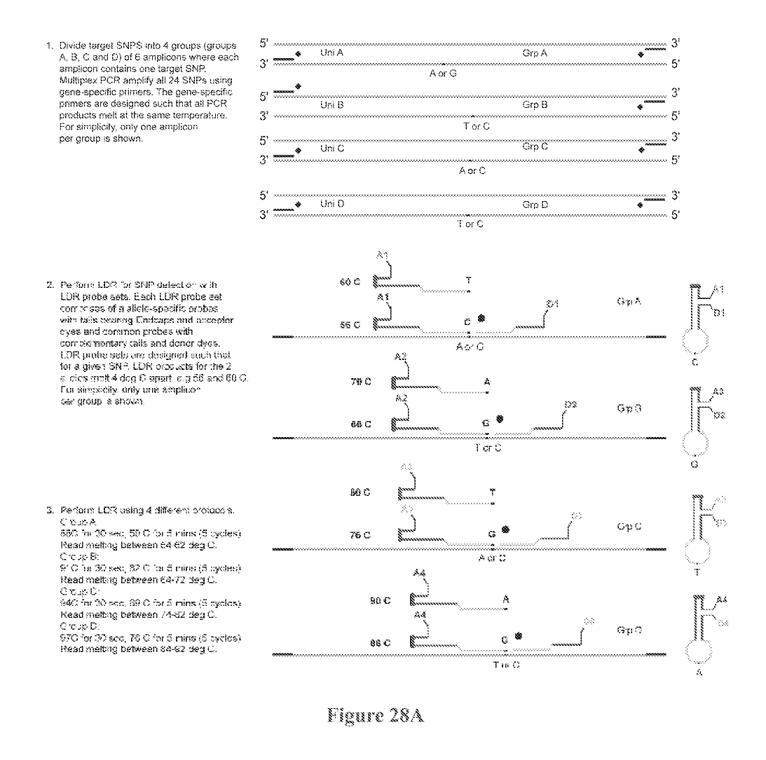

A further degree of multiplexing can be achieved using additional oligonucleotide probes having a distinct donor-acceptor FRET pair directed to a third set of SNPs in each group of PCR amplicons. After five rounds of ligation reaction cycling, the reaction is heated to 100° C. to completely denature the ligation products from the template and slowly cooled to measure the FRET signal. FIG. 27 shows an example of a melting curve generated using oligonucleotide probes with three distinct donor-acceptor FRET pairs. At the top left of the graph, the light blue line traces the ligation reaction cycling conditions and temperature ramp. At the bottom left of the graph, the purple, brown and orange lines represent the increasing fluorescence signal accumulated from all the probe ligation products bearing that donor-acceptor pair. On the right hand side of the graph, the purple, brown and orange lines show the melt curves measured for the three donor-acceptor FRET pairs. The red line represents the derivative of the purple melt curve, the green line represents the derivative of the brown melt curve, while the yellow line represents the derivative of the orange melt curve. The temperature of FRET detection and the resulting genotypes for Group A-D targets are summarized in the table accompanying the melt curve in FIG. 27.

In an alternative embodiment of the present method, the initial PCR phase can be designed such that the extension products have differing melting temperatures as shown in step 1 of FIG. 28B. In this embodiment, the extension products can be selectively denatured allowing for ligation to proceed sequentially on only the denatured extension products.

In this embodiment, the extension products differ in melting temperature as a result of their percent GC content. The universal tail portions of the extension products contain nucleotide sequences having increasing percent GC content from one pair of universal tails to the next pair. The melting temperatures of the extension products are determined by the sequence and the percent GC content of the target specific portion of the extension product as well as the sequence and the percent GC content of the 5′ and 3′ universal tail portions of the extension product.

In a preferred embodiment, the melting temperature of extension products generated using a first group of universal primer pair tail portions is different from the melting temperature of extension products generated using a second group of universal primer pair tail portions. Therefore, following the completion of the polymerase chain reaction cycles, the extension products, which are the targets for the subsequent ligase detection reaction phase, are denatured sequentially at different temperatures.

As shown in FIG. 28B, the target gene sequences to be amplified in the PCR phase can be grouped based on their melting temperatures. Group A is composed of targets having the most AT rich sequences. Primers amplifying Group A gene targets are designed to have universal primer tails that are significantly AT rich and allow the final PCR products to denature at 88° C. Likewise, Group B is composed of the second most AT rich target sequences and primers for initial amplification have universal primer tails that allow the final PCR products to denature at 91° C. Group C is composed of the second most GC rich target sequences and primers for initial amplification have universal primer tails that have medium GC clamps to allow the final PCR products to denature at 94° C. Finally, Group D is composed of the most GC rich target sequences and primers for initial amplification have universal primer tails having longer GC clamps to allow the final PCR products to denature at 97° C.

In this scenario, Group A amplification products are predominantly melted at 88° C. for 30 seconds, allowing for a Group A oligonucleotide probe group to anneal and ligate at 55° C. As shown in step 2 of FIG. 28B, the oligonucleotide probe group would consist of one first oligonucleotide probe having a 3′ target specific portion with a Tm of about 55° C., where the 3′ base is complementary to one of the SNPs, and a 5′ tunable probe portion containing an end-capped hairpin, which is tuned to have a Tm value of 56° C. with an acceptor group. The probe group would also consist of a second first oligonucleotide probe having a 3′ target specific portion with a Tm of about 55° C., where the 3′ base is complementary to the second SNP, and a 5′ tunable probe portion containing an end-capped hairpin, tuned to have a Tm value of 60° C. with the same acceptor group. The corresponding second oligonucleotide probe has a 5′ target specific portion and a tunable probe portion with a donor group at the 3′ end. If there is perfect complementarity at the junction between a first and second probe and the target, the two probes ligate together as shown in step 3 of FIG. 28B. Upon denaturing the ligation product from the target, the tunable probe portions of the first and second oligonucleotide probes form a double-stranded duplex at 56° C. or less, whereupon the donor and acceptor groups of the upstream and downstream probes are in close enough proximity allowing for a FRET to occur at 56° C. or less upon excitation of the donor fluorophore at the appropriate wavelength. The melting curve is determined around the 53° C.-63° C. range, and the target is genotyped as either homozygous for one allele, heterozygous, or homozygous for the second allele, in each case providing a distinct melting peak. The Group A oligonucleotide probes are designed to hybridize at 55° C., and also generate the melting curves for reading at 56° C. and 60° C. Similar FRET signals from ligation products within the same group (i.e. Group A products) are distinguished by the melting peaks of the first derivative of the melt curves, which differ by at least 4° C.

Following the completion of the ligase detection reaction for the Group A gene targets, the ligase detection reaction cycling is repeated and the denaturation and hybridization treatment temperatures are increased. Group B target amplification products are denatured at 91° C. for 30 seconds, allowing for Group B oligonucleotide probes to anneal and ligate at 62° C. The Group B oligonucleotide probes have tunable probes that are read at 66° C. and 70° C. Thus, the Group B ligation products will be formed and read at temperatures ranging from 63° C.-73° C. range.

For the next round of ligation detection reaction cycles, the temperature of the denaturation and hybridization treatments are further increased. Group C target amplification products are denatured at 94° C. for 30 seconds, allowing for Group C oligonucleotide probes to anneal and ligate at 69° C. The Group C oligonucleotide probes have tunable probes that are read at 76° C. and 80° C. Thus, the Group C ligation products will be formed and read at temperatures ranging from 73° C.-83° C.

For the next round of ligation detection reaction cycles, the temperature of the denaturation and hybridization treatments are further increased. Group D target amplification products are denatured at 97° C. for 30 seconds, allowing for Group D oligonucleotide probes to anneal and ligate at 76° C. The Group D oligonucleotide probes have tunable probes that are read at 86° C. and 90° C. Thus, the Group D ligation products will be formed and read at temperatures ranging from 83° C.-93° C.

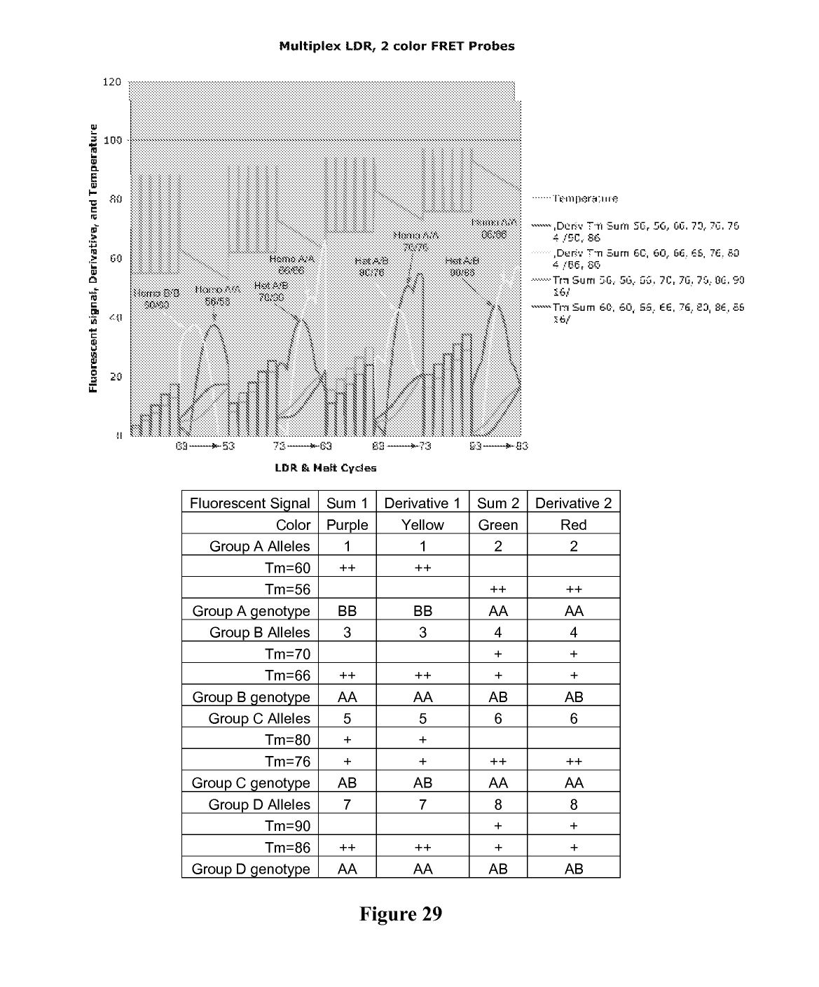

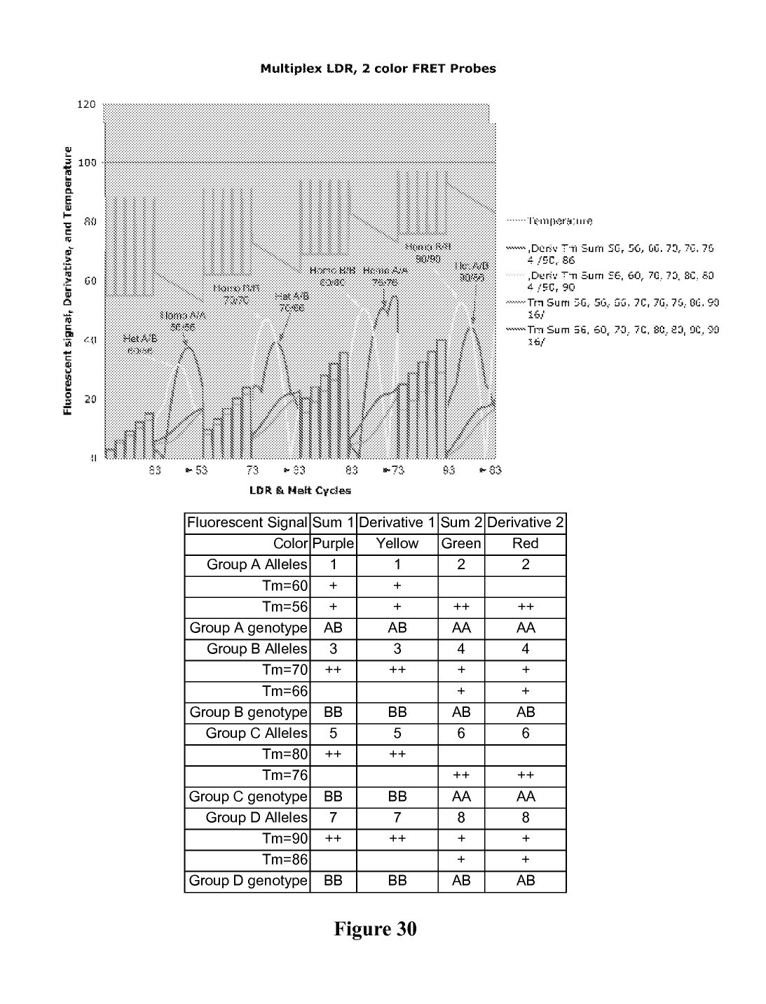

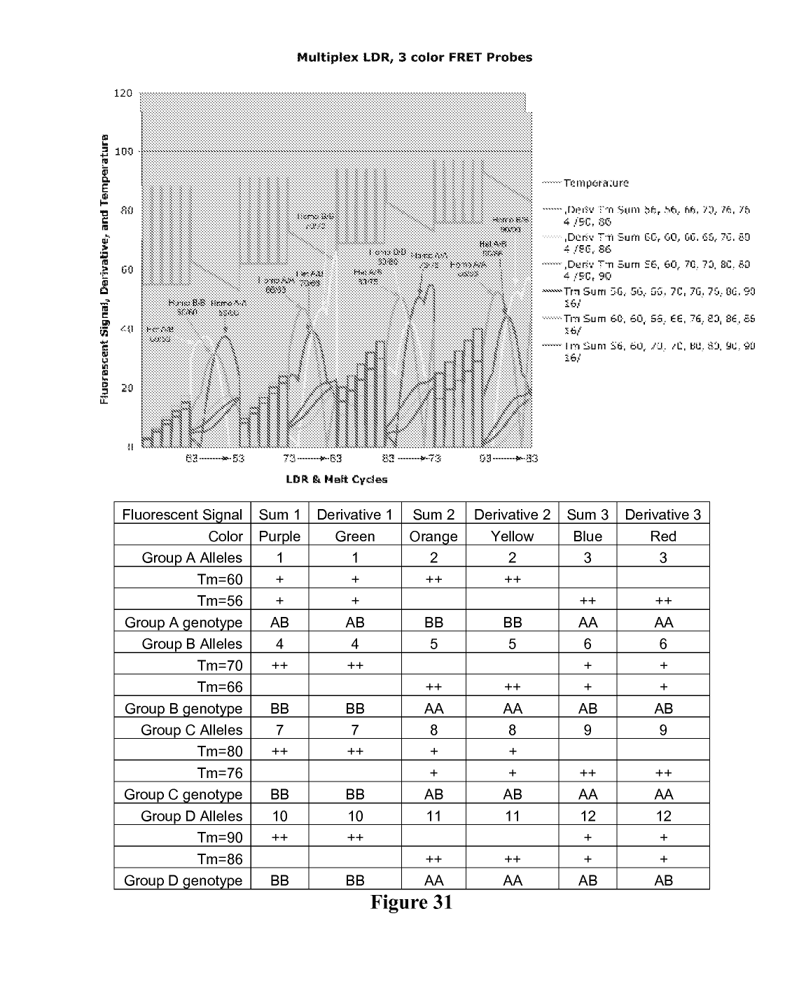

FIGS. 29-31 show typical melting curves calculated for this approach. In each of these figures, the light blue line corresponds to the temperature conditions during the ligation reaction cycles and FRET detection. First, five ligation reaction cycles are performed with denaturing temperature of 88° C. (only extension products that belong to Group A designed to melt at 88° C. are denatured) and the ligation temperature is 55° C. Thus, only the oligonucleotide probes designed to Group A amplicons will ligate with 100% efficiency. The higher melting oligonucleotide probes for group B amplicons (designed to melt at 91° C.) may also ligate, but at only 25% efficiency and thus contribute up to 25% of the FRET signal measured at the readout temperature range of 54-62° C. Likewise the even higher melting oligonucleotide probes designed for group C amplicons (designed to melt at 94° C.) may ligate at only 10% efficiency and contribute up to 10% of the FRET signal measured at the readout temperature range of 64-72° C. The highest melting probes designed for group D amplicons (designed to melt at 97° C.) should not contribute to the measured FRET signals. After the FRET signals are measured in the 54-62° C. range, five more ligation cycles are performed. This time the denaturation temperature is raised to 87° C., thus preferentially melting the group B amplicons. Likewise the ligation reaction annealing temperatures are now 62° C. After these five ligation reaction cycles are completed, the FRET readout is measured between 64 to 72° C. The next five ligation reaction cycles use a denaturation temperature of 94° C. and a ligation temperature of 69° C., followed by readout between 74-82° C. The final five ligation cycles use a denaturation temperature of 97° C., ligation temperature of 76° C. and FRET readout is measured between 84-92° C.

Accompanying each melt curve analysis of FIGS. 29-31 is a table summarizing the genotypes of the Group A-D targets as determined from the melting peaks of the derivative curves shown in the analysis.

In the preceding example, with each sequential round of ligase detection reaction cycling, the denaturation and hybridization temperatures are increased such that the hybridization temperatures of a first group of oligonucleotide probes to their target nucleic acid molecules is lower than the hybridization temperatures of a second group of oligonucleotide probes to their target nucleic acid molecules. Additionally, the 5′ endcapped hairpin tunable portions of the one or more first oligonucleotide probes in the first probe group have melting temperatures that are lower than the 5′ endcapped hairpin tunable portions of the one or more first oligonucleotide probes in the second probe group. In such an embodiment, the same FRET signals from ligation products from a first probe group are distinguished from the FRET signals for ligation products from a second probe group by the melting peaks of the first derivative of the melt curve which are at least 6° C. higher for the second group than for the first group. In this embodiment, the ligation products from the higher groups accumulate with each increasing round.

In an alternative embodiment, with each sequential round of ligase detection reaction cycling, the denaturation temperature is raised or increased while the hybridization temperature is lowered or decreased.

This design is based on the principle that the lower Tm tunable ligation products will not interfere with readout of the higher Tm tunable ligation products, so it does not matter if they accumulate when reading the latter groups. In this design, the ligation temperatures are flipped such that group A, B, C, and D probes ligate at 76° C., 69° C., 62° C., and 55° C., respectively (in contrast to 55° C., 62° C., 68° C., and 76° C.).

Consistent with this embodiment of the present invention, the hybridization temperatures of the first group of oligonucleotide probes to their target nucleic acid molecules is higher than the hybridization temperatures of the second group of oligonucleotide probes to their target nucleic acid molecules. In addition, the 5′ endcapped hairpin tunable portions of the one or more first oligonucleotide probes in the first probe group have melting temperatures that are lower than the 5′ endcapped hairpin tunable portions of the one or more first oligonucleotide probes in the second probe group. In such an embodiment, the same FRET signals from ligation products from a first probe group are distinguished from the FRET signals for ligation products from a second probe group by the melting peaks of the first derivative of the melt curve which are at least 6° C. higher for the second group than for the first group.

For the first round, Group A products denature at 88° C. for 30 seconds, allowing for Group A oligonucleotide probes to anneal and ligate at 76° C. The Group A probes have tunable portions that are read at 56° C. and 60° C. Thus, the Group A ligation products will be formed and read at temperatures ranging from 54° C.-62° C. Similar FRET signals from ligation products within the same group (i.e. Group A products) are distinguished by the melting peaks of the first derivative of the melt curves, which differ by at least 4° C.

For the next round, the temperature of the denaturation treatment and cycling conditions is a bit higher. Group B products denature at 91° C. for 30 seconds, allowing for Group B oligonucleotide probes to anneal and ligate at 69° C. The Group B probes have tunable portions that are read at 66° C. and 70° C. Thus, the Group B ligation products will be formed and read at temperatures ranging from 64° C.-72° C.

For the next round, the temperature of the denaturation treatment and cycling conditions is a bit higher. Group C products are denatured at 94° C. for 30 seconds, allowing for Group C oligonucleotide probes to anneal and ligate at 62° C. The Group C probes have tunable portions that are read at 76° C. and 80° C. Thus, the Group C ligation products will be formed and read at temperatures ranging from 74° C.-82° C.

For the next round, the temperature of the denaturation treatment and cycling conditions is a bit higher. Group D products denature at 97° C. for 30 seconds, allowing for Group D oligonucleotide probes to anneal and ligate at 55° C. The Group D probes have tunable portions that are read at 86° C. and 90° C. Thus, the Group D ligation products will be formed and read at temperatures ranging from 84° C.-92° C.

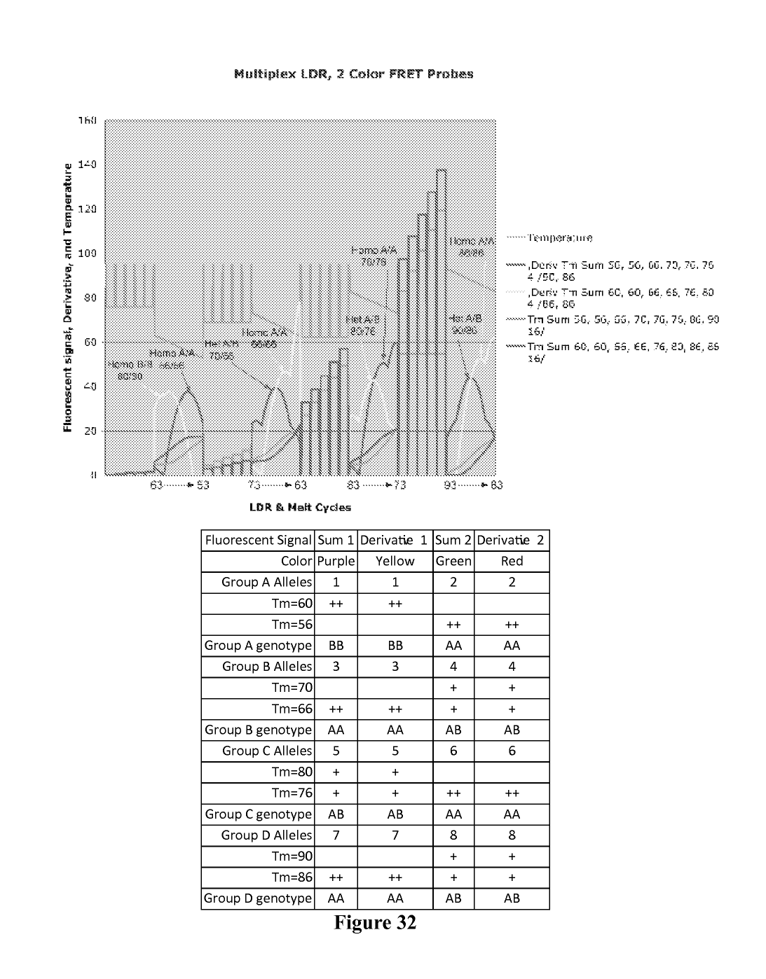

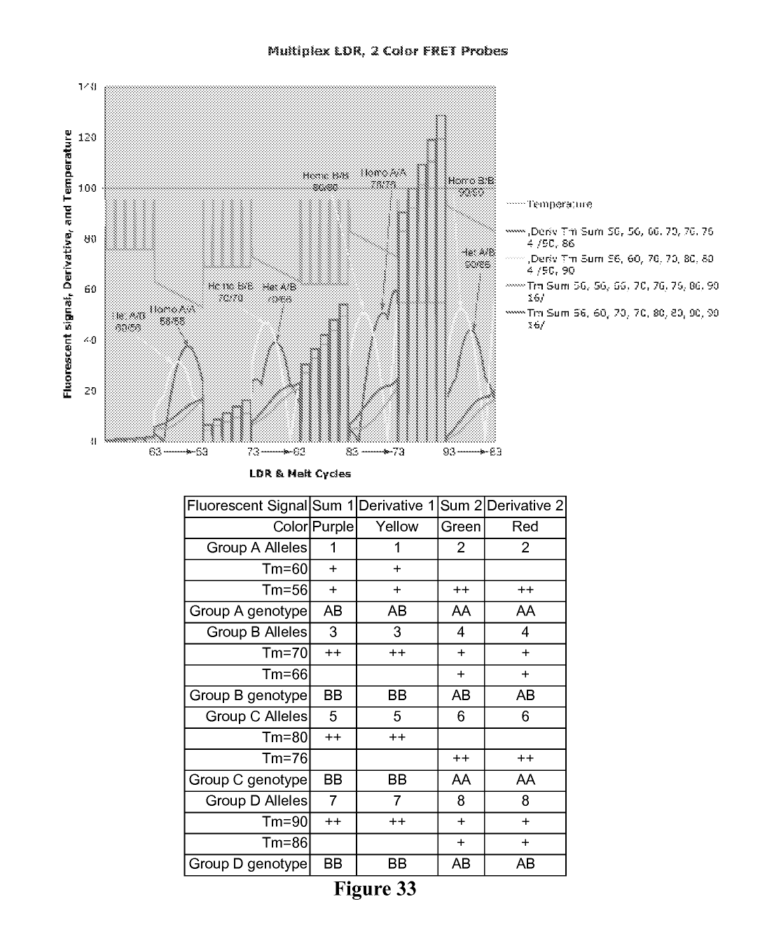

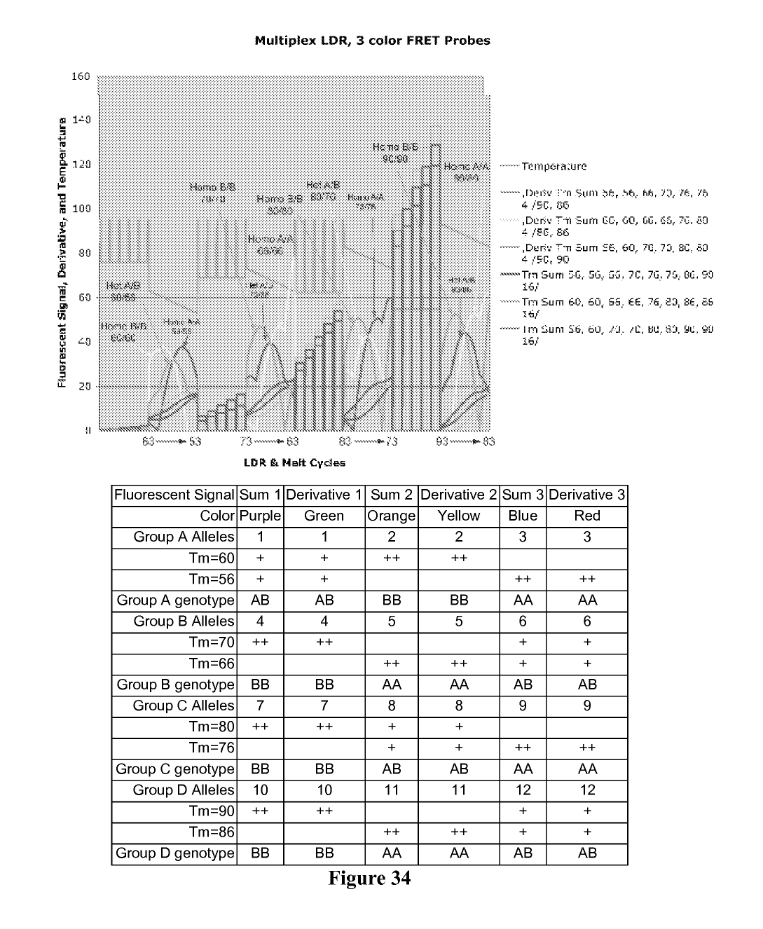

Examples of typical melt curves are shown in FIGS. 32-34. In these figures, note that the ligation temperatures (blue lines) which are shown in the context of the ligation reaction cycles are decreasing with each ligation reaction. However, the temperature at which the FRET signals are read subsequent to each set of LDR cycles is increasing.

Another aspect of the present invention involves a kit for identifying one or more of a plurality of target nucleic acid molecules in a sample using the methods of the present invention. This kit includes a ligase and a plurality of oligonucleotide probe sets. As described supra, each probe set is characterized by a first oligonucleotide probe having a target-specific portion and a tunable portion with an endcapped hairpin, and a second oligonucleotide probe comprising a target specific portion and a tunable portion, wherein one of the first and second oligonucleotide probes has an acceptor group and the other of the first and second probes has a donor group. The kit can also include a plurality of oligonucleotide primer sets suitable for amplification of the target nucleic acid molecules along with a polymerase. The oligonucleotide primers of a primer set can each include a target-portion and a universal tail portion and may be suitable for multiplex PCR amplification.

Another aspect of the present invention is directed to a method for detecting one or more of a plurality of target nucleic acid molecules in a sample. This method includes providing a sample potentially containing one or more target nucleic acid molecules and a plurality of oligonucleotide probe sets. Each probe set is characterized by a first oligonucleotide probe having a target-specific portion and a tunable portion with an endcapped hairpin and a second oligonucleotide probe having a target specific portion and a tunable portion, wherein one of the first and second oligonucleotide probes has an acceptor group and the other of the first and second probes has a donor group. The nucleotide sequence of the tunable portion of the first oligonucleotide probe in a probe set is complementary to the nucleotide sequence of the tunable portion of the second oligonucleotide probe in a probe set. The sample and the plurality of oligonucleotide probe sets are blended to form a hybridization mixture. The mixture is subjected to one or more hybridization cycles each cycle involving a denaturation and hybridization treatment. During the denaturation treatment any hybridized nucleic acid sequences are separated. During the hybridization treatment, the target-specific portions of a set of oligonucleotide probes hybridize to their respective target nucleotide sequences, if present in the sample, and the tunable portions of the set of oligonucleotide probes hybridize to each other to form an internally hybridized oligonucleotide probe set. The FRET between the donor and acceptor groups of each internally hybridized oligonucleotide probe set is detected, thereby indicating the presence of a target nucleic acid sequences in the sample.

FIG. 3 depicts one structural embodiment of the oligonucleotide probes used in accordance with this aspect of the invention. In the embodiment shown, a first oligonucleotide probe in a probe set has the Tm master tunable end-capped hairpin portion, tunable probe portion and the donor dye attached to the 5′-end of an oligonucleotide that is complementary to the target sequence. The second oligonucleotide probe in a probe set has the complementary tunable probe portion and the acceptor dye attached to the 3′-end of an oligonucleotide that is complementary to the target sequence. In an alternative structure of the oligonucleotide probes, the first oligonucleotide probe in a probe set has the Tm master tunable endcapped hairpin portion, tunable probe portion and an acceptor dye attached to the 5′-end of an oligonucleotide that is complementary to the target sequence. The second oligonucleotide probe in the probe set has the complementary tunable probe portion and the donor dye attached to the 3′-end of an oligonucleotide that is complementary to the target sequence. In another alternative structure of the oligonucleotide probes, the first oligonucleotide probe in a probe set has a tunable probe portion and the donor dye attached to the 5′-end of an oligonucleotide that is complementary to the target sequence. The second oligonucleotide probe in the probe set has the complementary tunable probe portion with the Tm master tunable endcapped hairpin portion and the acceptor dye attached to the 3′-end of an oligonucleotide that is complementary to the target sequence, In yet another alternative design, the first oligonucleotide probe in a probe set has a tunable probe portion and the acceptor dye attached to the 5′-end of an oligonucleotide that is complementary to the target sequence. The second oligonucleotide probe in the probe set has the complementary tunable probe portion with the Tm master tunable endcapped hairpin portion and the donor dye attached to the 3′-end of an oligonucleotide that is complementary to the target sequence. Regardless of the probe design structure, the second oligonucleotide probe hybridizes 2-3 bases downstream of the first oligonucleotide probe as shown in FIG. 3.

The target-specific portions in any of the above described oligonucleotides probes in a probe set and the tunable portions of the oligonucleotide probes in a probe set can have the same or similar melting temperature. When the oligonucleotide probes in a set hybridize to a target sequence, the donor and acceptor groups are brought in close proximity to each other, producing a quantitative FRET signal. The amount of signal produced is proportional to the amount of target present in the sample. The second oligonucleotide probes can have a blocking group at their 3′ ends to prevent extension when bound to the target. The first oligonucleotide probes have a donor or acceptor group on their 3′ end already blocking such extension.

The oligonucleotide probes used in accordance with this aspect of the present invention share many attributes of the oligonucleotide probes describes supra. For example, the probe sets can be formed of ribonucleotides, deoxynucleotides, modified ribonucleotides, modified deoxyribonucleotides, peptide nucleotide analogues, modified peptide nucleotide analogues, modified phosphate-sugar-backbone oligonucleotides, nucleotide analogs, and mixtures thereof. Additionally, the acceptor and donor fluorophore groups, as well as, the endcapped hairpin portions of the oligonucleotide probes described supra are suitable for use in this aspect of the invention.

Multiple target sequences can be detected under similar hybridization conditions when the oligonucleotide probes have different acceptor-donor groups and multiple FRET signals are detected. In another embodiment, multiple target sequences can be detected under different hybridization conditions using probes that have the same acceptor-donor groups, but different melting temperatures. In this embodiment, the hybridization step is repeated one or more times. With each repeat, the temperature is increased to allow for oligonucleotide probes having higher melting temperatures to hybridize to their respective target sequence. FRET signal can be detected following each round of hybridization. In a preferred embodiment, target detection can be further multiplexed by using oligonucleotide probes having similar melting temperatures and different acceptor-donor groups in combination with oligonucleotide probes having differing melting temperatures and the same acceptor-donor group.

In accordance with this aspect of the present invention, it may be desirable to simultaneously amplify the target sequence. In this embodiment, a plurality of oligonucleotide primer sets and a DNA polymerase is provided. The sample, the plurality of oligonucleotide primer sets, the plurality of oligonucleotide probe sets, and the DNA polymerase are blended to form a polymerase chain reaction mixture. The mixture is subjected to one or more polymerase chain reaction cycles, each cycle comprising a denaturation and a hybridization treatment. During the denaturation treatment, any hybridized nucleic acid sequences are separated. During the hybridization treatment, the oligonucleotide primer sets hybridize to their respective target nucleotide sequences, if present in the sample, and extend to form extension products. In addition, the target-specific portions of a set of oligonucleotide probes hybridize to their respective target nucleotide sequences, if present in the sample, and the tunable portions of the set of oligonucleotide probes hybridize to each other to form an internally hybridized oligonucleotide probe set. At the completion of each polymerase chain reaction cycle the fluorescence resonance energy transfer (FRET) between the donor and acceptor groups of each internally hybridized oligonucleotide probe set is detected.

The oligonucleotide primers of a primer set used for target sequence amplification each comprise a target-specific portion and a universal primer pair tail. The plurality of oligonucleotide primer sets form a plurality of oligonucleotide primer groups. Each primer group is characterized by oligonucleotide primer sets having the same or similar melting temperature. Likewise, the plurality of oligonucleotide probe sets form a plurality of oligonucleotide probe groups. Each probe group is characterized by oligonucleotide probe sets having the same or similar melting temperature.

As described supra, multiple target sequences can be detected under similar denaturation and hybridization conditions when the oligonucleotide probes have different acceptor-donor groups and the multiple FRET signals are detected with each polymerase chain reaction cycle. In another embodiment, multiple target sequences can be detected using probes that have the same acceptor-donor groups, but different melting temperatures. In this embodiment, when the polymerase chain reaction cycles are repeated, the denaturation and hybridization treatment temperatures are increased to allow for oligonucleotide primer and oligonucleotide probe sets having higher melting temperatures to hybridize to their respective target sequences. FRET signal can be detected following each round of denaturation and hybridization. In a preferred embodiment, target detection can be further multiplexed by using oligonucleotide primer and probe groups having similar melting temperatures and different acceptor-donor groups in combination with oligonucleotide primer and probe groups having differing melting temperatures and the same acceptor-donor group.

In one embodiment, this aspect of the present invention can be used to quantitatively measure one or more of the plurality of target nucleic acid molecules having a plurality of sequence differences present in a sample. To achieve a quantitative measure of the target nucleic acid molecules, a known amount of one or more marker target nucleic acid molecules along with a plurality of marker-specific oligonucleotide primer sets are provided along with the sample. Each primer of the primer set is characterized by a marker-specific target portion and a universal primer tail. In addition, a plurality of marker-specific oligonucleotide probe sets is also provided. Each probe set is characterized by a first oligonucleotide probe comprising a marker target-specific portion and a tunable portion and a second oligonucleotide probe having a marker target specific portion and a tunable portion containing an endcapped hairpin, wherein one of the first and second oligonucleotide probes has an acceptor group and the other of the first and second probes has a donor group. The tunable portions of each probe set have a unique melting temperature. The marker target nucleotide sequences and the plurality of marker-specific oligonucleotide primers sets are mixed with the sample to form the polymerase chain reaction mixture, while the plurality of marker-specific oligonucleotide probe sets are mixed with the plurality of oligonucleotide probe sets to form the hybridization mixture.

The FRET of the marker-specific oligonucleotide probe sets is detected during each polymerase chain reaction cycle. A comparison of the amount of FRET generated from the known amount of marker target nucleotide sequences with the amount of FRET generated from the target nucleotide sequences provides a quantitative measure of the target nucleic acid molecule.

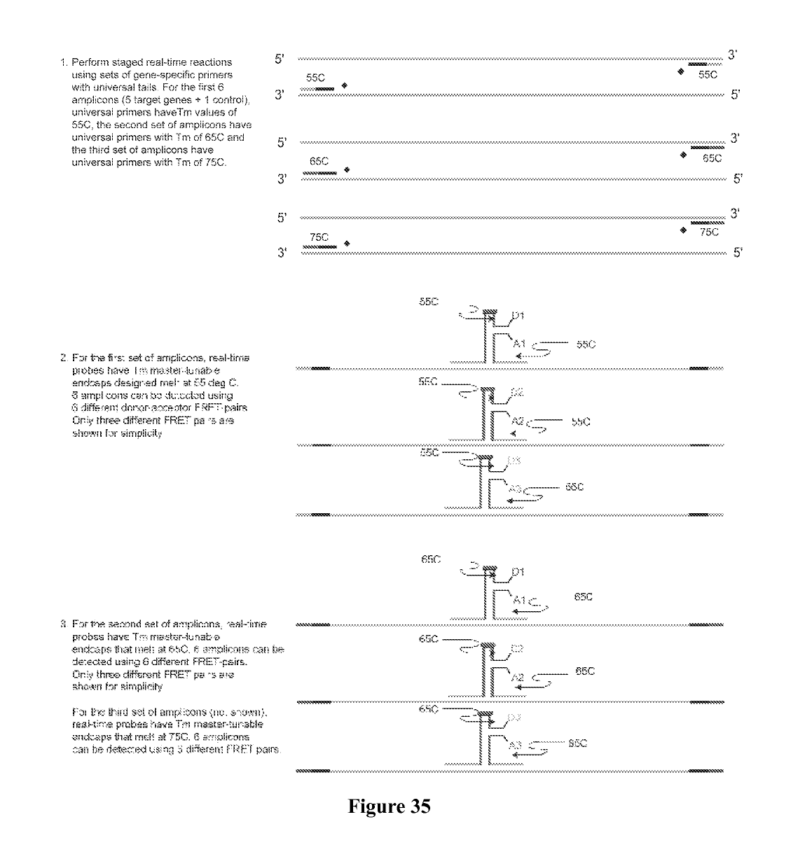

FIG. 35 is a flow diagram of an experiment where the tunable readout of the relative level of expression of fifteen test genes and three control genes is carried out using the oligonucleotide probes in accordance with this aspect of the present invention. The fifteen test genes and three control genes are grouped together into three groups of six genes each (five test genes and one control gene per group). The starting sample can be DNA or RNA that is reverse transcribed to generate cDNA. When using RNA, an enzyme such as Tth polymerase, which has both reverse transcription activity, as well as DNA polymerase activity can be used.

Initial experiments are performed to determine the approximate order of amplification (reflecting the relative levels of expression) of the genes of interest. The three groups of six genes are chosen such that the genes in Group A are generally expressed at the highest levels among the target genes. Thus, Group A contains the fastest amplifying amplicons. For this group, gene-specific PCR primers are designed to have universal primer pair A tails. Universal primers A have Tm values of 55° C. (step 1 of FIG. 35).

The next group of genes (Group B) contains genes that generally have a medium level of expression. For this group, gene-specific PCR primers are designed to have universal primer pair B tails. Universal primers B have Tm values of 65° C. (step 1 of FIG. 35).

Finally, the third group of genes (Group C) contains those genes that generally have a low level of expression. For this group, gene-specific PCR primers are designed to have universal primer pair C tails. Universal primers C have Tm values of 75° C. (step 1 of FIG. 35).

Oligonucleotide probes with distinct donor-acceptor FRET pairs are used for each of the genes in Group A (6 different colors). These Group A (fastest amplifying group) probe pairs bind well to the target DNA at 55° C., but not well at 65° C. (step 2 of FIG. 35). Each downstream probe has a 3′ target specific portion and an additional tunable probe portion which is tuned to have a Tm value of 55° C. with an acceptor group at the 5′ end. Each upstream probe has a 5′ target specific portion and an additional tunable probe portion containing an endcapped hairpin with a donor group at the 3′ end. This upstream tunable probe portion forms a double-stranded region to the tunable probe portion of the downstream primer at 55° C. only when the upstream and downstream probes are brought in proximity to each other by binding to the same target strand. This results in the donor and acceptor groups of the upstream and downstream probes to be in close enough proximity allowing for a FRET to occur at 55° C. upon excitation of the donor fluorophore at the appropriate wavelength. Increasing the temperature to 65° C. significantly reduces or eliminates FRET signal from these probes due to (i) melting of the upstream probe from the target, (ii) melting of the downstream probe from the target, (iii) melting of the tunable probe portion containing an endcapped hairpin, and (iv) melting of the double-stranded region formed between the upstream and downstream probes to each other.

Oligonucleotide probe pairs for genes in Group B (medium amplifying group) bind well to the target DNA at 65° C., but not well at 75° C. (step 3 of FIG. 35). These pairs are similar to Group A above, except the tunable probe portion containing the endcapped hairpin, is tuned to have a Tm value of 65° C., and the double-stranded region where the two probes bind each other is also tuned to have a Tm value of 65° C.

Group C (slowest amplifying group) oligonucleotide probe pairs bind well to the target DNA at 75° C., but not so well at 85° C. These pairs are similar to Group A above, except the tunable probe portion containing the endcapped hairpin, is tuned to have a Tm value of 75° C., and the double-stranded region where the two probes bind each other is also tuned to have a Tm value of 75° C.

For measurement of gene expression, all 18 sets of gene specific primer pairs, all three sets of universal primers and all 18 sets of oligonucleotide probes are used simultaneously. The cycling conditions for the first round of PCR amplification include denaturation at 95° C. for 30 seconds; primer binding and extension and probe binding and measuring tunable FRET signal at 55° C. for 1 minute; and completion of primer extension at 72° C. for 1-2 minutes. These thermal cycling conditions are repeated until the FRET signal for the control gene in Group A is measurable above the threshold. If present, the five target genes in Group A will also provide measurable real-time FRET signal above threshold.

PCR amplification is repeated with cycles of 95° C. (30 seconds, for denaturation); 65° C. (1 minute for primer binding and extension, and for oligonucleotide probe binding and measuring tunable FRET signal); 72° C. (1-2 minutes to complete primer extension). Repeat these thermal cycling conditions until the FRET signal for the control gene in Group B is measurable above the threshold. If present, the five target genes in Group B will also provide measurable real-time FRET signal above threshold.

Products from the Group A amplicons will no longer amplify efficiently at 65° C. (their universal primers, with Tm values of 55 ° C., are too short). Further, signal from the 55° C. real-time probes will be significantly reduced at 65° C., so the predominant FRET signals from these cycles will be from Group B amplicons.

PCR amplification is repeated again using cycles of 95° C. (30 seconds, for denaturation); 75° C. (1 minute for primer binding and extension, and for probe binding and measuring tunable FRET signal); 75° C. (1-2 minutes to complete primer extension). These thermal cycling conditions are repeated until the FRET signal for the control gene in Group C is measurable above the threshold. If present, the five target genes in Group C will also provide measurable real-time FRET signal above threshold.

The products from the group A and B amplicons will no longer amplify efficiently at 75° C. (their universal primers, with Tm values of 55 and 65 ° C. respectively, are too short). Further, signal from the 55° C. and 65° C. real-time probes will be significantly reduced at 75° C., so the predominant FRET signals from these cycles will be from Group C amplicons.

The advantage of using tunable oligonucleotide probes for real-time detection is that the probe-FRET signal is based on the tunable portion of the probes, and not on the Tm of the probes hybridizing to the target. Thus, this approach can tolerate sequence variation in the target and provides considerable advantage in correctly identifying the target(s) compared with other multiplexed approaches.

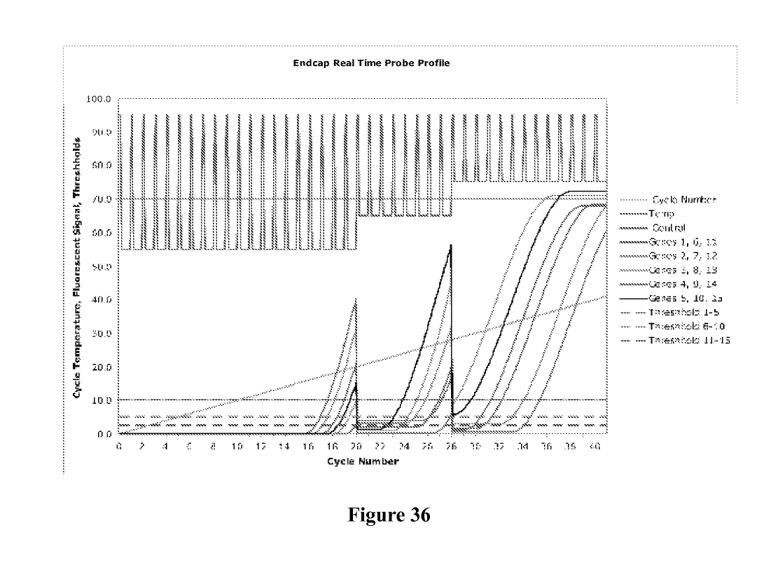

FIG. 36 shows the cycle threshold (Ct) curves for relative levels of gene expression measured using oligonucleotide probes of the present invention. The red line at top traces the cycling conditions during the reaction. The fastest amplifying targets (Group A) have Ct values between 14 and 20 cycles (threshold denoted by dotted red line). Group B amplicons have Ct values between 20 and 28 (threshold denoted by dotted grey line), while Group C amplicons have Ct values higher than 28 (threshold denoted by dotted black line). The threshold signal for each group is higher than the previous group, because the signal for a particular color is accumulating for all three groups form the first cycle.

Quantitation of the relative gene expression shown in FIG. 36 can be calculated using the Comparative C_{T} Method. The Comparative C_{T} method uses arithmetic formulas to achieve determine expression levels. A detailed description of relative gene expression quantitation is provided in the ABI Prism 7700 Sequence Detection System User Bulletin #2, which is hereby incorporated by reference in its entirety.

A final aspect of the present invention relates to a kit for carrying out this method of the present invention. This kit includes the plurality of oligonucleotide probe sets characterized by a first oligonucleotide probe comprising a target-specific portion and a tunable portion and a second oligonucleotide probe having a target specific portion and a tunable portion with an endcapped hairpin, wherein one of the first and second oligonucleotide probes has an acceptor group and the other of the first and second probes has a donor group. The kit may also contain a plurality of oligonucleotide primer sets and a DNA polymerase. In a preferred embodiment, the oligonucleotide primers in a primer set comprises a target-specific portion and a universal tail portion.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

What is claimed:

1. A method for identifying one or more of a plurality of target nucleic acid molecules in a sample, said method comprising:
providing a sample potentially containing one or more target nucleic acid molecules;
providing a plurality of oligonucleotide probe sets, each set characterized by (a) a first oligonucleotide probe, comprising a target-specific portion and a tunable portion with an endcapped hairpin and (b) a second oligonucleotide probe comprising a target specific portion and a tunable portion, wherein one of the first and second oligonucleotide probes has an acceptor group and the other of the first and second probes has a donor group;
providing a ligase;
blending the sample, the plurality of oligonucleotide probe sets, and the ligase to form a ligase detection reaction mixture;
subjecting the ligase detection reaction mixture to one or more ligase detection reaction cycles, each cycle comprising a denaturation treatment, wherein any hybridized oligonucleotides are separated from the target nucleic acid sequences, and a hybridization treatment, wherein the set of oligonucleotide probes hybridize in a base-specific manner to their respective target nucleotide sequences, if present in the sample, and ligate to one another to form a ligation product containing the tunable portions, the endcapped hairpin, the target-specific portions, the acceptor group, and the donor group;
subjecting the ligation products to conditions effective to permit hybridization of the tunable portions of the ligation product to one another to form an internally hybridized ligation product; and
detecting the fluorescence resonance energy transfer (FRET) between the donor and acceptor groups of the internally hybridized ligation product, thereby indicating the presence of a target nucleic acid molecule in the sample.

2. The method according to claim 1, wherein the oligonucleotide probe sets are suitable for ligation together at a ligation junction when hybridized adjacent to one another on a corresponding target nucleotide sequence due to perfect complementarity at the ligation junction, but, when the oligonucleotide probe sets are hybridized to any other nucleotide sequence present in the sample, have a mismatch at a base at the ligation junction which interferes with such ligation.

3. The method according to claim 1, wherein multiple allele differences at one or more nucleotide positions in a single target nucleic acid molecule or multiple allele differences at one or more nucleotide positions in multiple target nucleic acid molecules are distinguished, the oligonucleotide probe sets forming a plurality of oligonucleotide probe groups, each group comprised of one or more oligonucleotide probe sets designed for distinguishing multiple allele differences at a single nucleotide position, wherein, in the oligonucleotide probes of each group, the second oligonucleotide probes have a common target-specific portion, and the first oligonucleotide probes have differing target-specific portions which hybridize to a given allele in a base-specific manner and differing endcapped hairpin tunable probe portions, wherein, in said detecting, the FRET signals of internally hybridized ligation products from each probe set within each probe group, are detected, thereby indicating the presence, in the sample, of one or more alleles at one or more nucleotide position in one or more target nucleotide sequences.

4. The method according to claim 3, wherein the endcapped hairpin tunable portions of the one or more first oligonucleotide probes in a probe group have melting temperatures which differ by at least 4° C.

5. The method according to claim 3, wherein the endcapped hairpin tunable portions of the one or more first oligonucleotide probes in a first probe group have melting temperatures which differ by at least 6° C. from the one or more first oligonucleotide probes in a second probe group.

6. The method according to claim 3, wherein the endcapped hairpin tunable portions of the one or more first oligonucleotide probes in a probe group have the same acceptor group.

7. The method according to claim 3, wherein the target-specific portions of the oligonucleotide probes in a probe group have a melting temperature that is different than the melting temperature of the tunable portions of the oligonucleotide probes in a probe group.

8. The method according to claim 3, wherein probe groups having similar endcapped hairpin tunable portion melting temperatures have different acceptor-donor groups.

9. The method according to claim 1, wherein said detecting comprises:
performing a melt curve analysis to detect a plurality of internally hybridized ligation product.

10. The method according to claim 9, wherein the plurality of internally hybridized ligation products are detected at one or more FRET signals.

11. The method according to claim 9, wherein the plurality of internally hybridized ligation products with the same FRET signals are distinguished by melting peaks of a first derivative of the melt curve.

12. The method according to claim 11, wherein the same FRET signals from ligation products within the same probe group are distinguished by the melting peaks of a first derivative of the melt curve, which differ by at least 4° C.

13. The method according to claim 11, wherein the same FRET signals from ligation products from different probe groups are distinguished by the melting peaks of a first derivative of the melt curve, which differ by at least 6° C.

14. The method according to claim 1, wherein the donor group of the oligonucleotide probe is selected from the group consisting of Alexa Fluor 350, Marina Blue, Pacific Orange, Alexa Fluor 405, Pacific Blue, Alexa Fluor 430, Fluorescein and it's derivatives, Alexa Fluor 488, Oregon Green 488, Alexa Fluor 500, Alexa Fluor 514, Oregon Green 514, Alexa Fluor 532, Alexa Fluor 546, Cy3, Alexa Fluor 555, Tetramethylrhodamine and it's derivatives, Alexa Fluor 568, Cy 3.5, Alexa Fluor 594, Texas Red, Alexa Fluor 610, Alexa Fluor 633, Cy 5, Alexa Fluor 647, Alexa Fluor 660, Alexa Fluor 680, Cy 5.5, and Alexa Fluor 700.

15. The method according to claim 1, wherein the acceptor group of the oligonucleotide probe is selected from the group consisting of Marina Blue, Pacific Orange, Alexa Fluor 405, Pacific Blue, Alexa Fluor 430, Fluorescein and it's derivatives, Alexa Fluor 488, Oregon Green 488, Alexa Fluor 500, Alexa Fluor 514, Oregon Green 514, Alexa Fluor 532, Alexa Fluor 546, Cy3, Alexa Fluor 555, Tetramethylrhodamine and it's derivatives, Alexa Fluor 568, Cy 3.5, Alexa Fluor 594, Texas Red, Alexa Fluor 610, Alexa Fluor 633, Cy 5, Alexa Fluor 647, Alexa Fluor 660, Alexa Fluor 680, Cy 5.5, Alexa Fluor 700, and Alexa Fluor 750.

16. The method according to claim 1, wherein the endcapped hairpin portion of the first oligonucleotide probe comprises an aromatic endcap or an aliphatic endcap.

17. The method according to claim 1 further comprising:
providing a plurality of oligonucleotide primer sets and a DNA polymerase;
blending the sample, the plurality of oligonucleotide primers, and the polymerase to form a polymerase chain reaction mixture prior to forming said ligase detection reaction mixture; and
subjecting the polymerase chain reaction mixture to one or more polymerase chain reaction cycles comprising a denaturation treatment, wherein hybridized nucleic acid sequences are separated, a hybridization treatment, wherein the primers hybridize to their complementary target-specific portions, and an extension treatment, wherein the hybridized primers are extended to form extension products, the ligase detection reaction mixture being formed by blending the extension products rather than the sample.

18. A method for identifying one or more of a plurality of target nucleic acid molecules in a sample, said method comprising:
providing a sample potentially containing one or more target nucleic acid molecules;
providing a plurality of oligonucleotide primer sets wherein each oligonucleotide primer of a primer set comprises a target-portion and a universal tail portion;
providing a DNA polymerase;
blending the sample, the plurality of oligonucleotide primers, and the polymerase to form a polymerase chain reaction mixture;
subjecting the polymerase chain reaction mixture to one or more polymerase chain reaction cycles comprising a denaturation treatment, wherein hybridized nucleic acid sequences are separated, a hybridization treatment, wherein the primers hybridize to their complementary target-specific portions, and an extension treatment, wherein the hybridized primers are extended to form extension products;
providing a plurality of oligonucleotide probe sets, each set characterized by (a) a first oligonucleotide probe, comprising a target-specific portion and a tunable portion with an endcapped hairpin and (b) a second oligonucleotide probe comprising a target specific portion and a tunable portion, wherein one of the first and second oligonucleotide probes has an acceptor group and the other of the first and second probes has a donor group;
providing a ligase;
blending the sample containing the extension products, the plurality of oligonucleotide probe sets, and the ligase to form a ligase detection reaction mixture;
subjecting the ligase detection reaction mixture to one or more ligase detection reaction cycles, each cycle comprising a denaturation treatment, wherein any hybridized oligonucleotides are separated from the target nucleic acid sequences, and a hybridization treatment, wherein a set of oligonucleotide probes hybridize in a base-specific manner to their respective target nucleotide sequences, if present in the sample, and ligate to one another to form a ligation product containing the tunable portions, the endcapped hairpin, the target-specific portions, the acceptor group, and the donor group;
subjecting the ligation products to conditions effective to permit hybridization of the tunable portions of the ligation product to one another to form an internally hybridized ligation product; and
detecting the fluorescence resonance energy transfer (FRET) between the donor and acceptor groups of the internally hybridized ligation product, thereby indicating the presence of a target nucleic acid molecule in the sample.

19. The method according to claim 18, wherein the oligonucleotide probe sets are suitable for ligation together at a ligation junction when hybridized adjacent to one another on a corresponding target nucleotide sequence due to perfect complementarity at the ligation junction, but, when the oligonucleotide probe sets are hybridized to any other nucleotide sequence present in the sample, have a mismatch at a base at the ligation junction which interferes with such ligation.

20. The method according to claim 18, wherein multiple allele differences at one or more nucleotide positions in a single target nucleic acid molecule or multiple allele differences at one or more nucleotide positions in multiple target nucleic acid molecules are distinguished, the oligonucleotide probe sets forming a plurality of oligonucleotide probe groups, each group comprised of one or more oligonucleotide probe sets designed for distinguishing multiple allele differences at a single nucleotide position, wherein, in the oligonucleotide probes of each group, the second oligonucleotide probes have a common target-specific portion, and the first oligonucleotide probes have differing target-specific portions which hybridize to a given allele in a base-specific manner and differing endcapped hairpin tunable probe portions, wherein, in said detecting, the FRET signals of internally hybridized ligation products from each probe set within each probe group, are detected, thereby indicating the presence, in the sample, of one or more alleles at one or more nucleotide position in one or more target nucleotide sequences.

21. The method according to claim 20, wherein the endcapped hairpin tunable portions of the one or more first oligonucleotide probes in a probe group have melting temperatures which differ by at least 4° C.

22. The method according to claim 20, wherein the endcapped hairpin tunable portions of the one or more first oligonucleotide probes in a first probe group have melting temperatures which differ by at least 6° C. from the one or more first oligonucleotide probes in a second probe group.

23. The method according to claim 20, wherein the endcapped hairpin tunable portions of the one or more first oligonucleotide probes in a probe group have the same acceptor group.

24. The method according to claim 20, wherein the target-specific portions of the oligonucleotide probes in a probe group have a melting temperature that is different than the melting temperature of the tunable portions of the oligonucleotide probes in a probe group.

25. The method according to claim 20, wherein probe groups having similar endcapped hairpin tunable portion melting temperatures have different acceptor-donor groups.

26. The method according to claim 18, wherein said detecting further comprises:
performing a melt curve analysis to detect a plurality of internally hybridized ligation product.

27. The method according to claim 26, wherein the plurality of internally hybridized ligation products are detected at one or more FRET signals.

28. The method according to claim 27, wherein the plurality of internally hybridized ligation products with the same FRET signals are distinguished by melting peaks of a first derivative of the melt curve.

29. The method according to claim 28, wherein the same FRET signals from ligation products within the same probe group are distinguished by the melting peaks of a first derivative of the melt curve, which differ by at least 4° C.

30. The method according to claim 28, wherein the same FRET signals from ligation products from different probe groups are distinguished by the melting peaks of a first derivative of the melt curve, which differ by at least 6° C.

31. The method according to claim 18, wherein the extension product sequences differ in melting temperatures.

32. The method according to claim 18, wherein the extension product sequences contain a 5′ universal tail portion, a target portion, and a 3′ universal tail portion.

33. The method according to claim 32, wherein the universal tail portions of the extension products contain nucleotide sequences with increasing % GC content from one pair of universal tails to the next pair.

34. The method according to claim 33, wherein the extension products have different melting temperatures determined by the sequence and % GC content of the target specific portion of the extension products as well as the sequence and % GC content of the 5′ and 3′ universal tail portions of the extension products.

35. The method according to claim 34, wherein the melting temperature of extension products generated using a first group of universal primer pair tail portions is different from the melting temperature of extension products generated using a second group of universal primer pair tail portions.

36. The method according to claim 18, further comprising:
repeating said subjecting the ligase detection reaction mixture to one or more ligase detection reaction cycles, wherein each time said subjecting the ligase detection reaction mixture to one or more reaction cycles is repeated, the denaturation and hybridization treatment temperature increases;
subjecting the ligation products formed from said repeating, to conditions effective to permit hybridization of the tunable portions of the ligation products to one another to form internally hybridized ligation products; and
detecting the fluorescence resonance energy transfer (FRET) between the donor and acceptor groups of the internally hybridized ligation products, thereby indicating the presence of a target nucleic acid molecule in the sample.

37. The method according to claim 36, wherein the hybridization temperatures of a first group of oligonucleotide probes to their target nucleic acid molecules is lower than the hybridization temperatures of a second group of oligonucleotide probes to their target nucleic acid molecules.

38. The method according to claim 37, wherein the endcapped hairpin tunable portions of the one or more first oligonucleotide probes in the first probe group have melting temperatures that are lower than the endcapped hairpin tunable portions of the one or more first oligonucleotide probes in the second probe group.

39. The method according to claim 36, wherein said detecting further comprises:
performing a melt curve analysis to detect a plurality of internally hybridized ligation product.

40. The method according to claim 39, wherein the plurality of internally hybridized ligation products are detected at one or more FRET signals.

41. The method according to claim 39, wherein the plurality of internally hybridized ligation products with the same FRET signals are distinguished by melting peaks of a first derivative of the melt curve.

42. The method according to claim 41, wherein the same FRET signals from ligation products within the same probe group are distinguished by the melting peaks of the first derivative of the melt curve, which differ by at least 4° C.

43. The method according to claim 41, wherein the same FRET signals from ligation products from the first probe group are distinguished from the FRET signals for ligation products from the second probe group, by the melting peaks of a first derivative of the melt curve, wherein the melting peak for the second group is at least 6° C. higher than for the first group.

44. The method according to claim 18, further comprising:
repeating said subjecting the ligase detection reaction mixture to one or more reaction cycles, wherein each time said subjecting the ligase detection reaction mixture to one or more reaction cycles is repeated, the denaturation treatment temperature increases and hybridization treatment temperature decreases;
subjecting the ligation products formed from said repeating, to conditions effective to permit hybridization of the tunable portions of the ligation products to one another to form internally hybridized ligation products; and
detecting the fluorescence resonance energy transfer (FRET) between the donor and acceptor groups of the internally hybridized ligation products, thereby indicating the presence of a target nucleic acid molecule in the sample.

45. The method according to claim 44, wherein the hybridization temperatures of a first group of oligonucleotide probes to their target nucleic acid molecules is higher than the hybridization temperatures of a second group of oligonucleotide probes to their target nucleic acid molecules.

46. The method according to claim 44, wherein the endcapped hairpin tunable portions of the one or more first oligonucleotide probes in the first probe group have melting temperatures that are lower than the endcapped hairpin tunable portions of the one or more first oligonucleotide probes in the second probe group.

47. The method according to claim 44, wherein said detecting further comprises:
performing a melt curve analysis to detect a plurality of internally hybridized ligation product.

48. The method according to claim 47, wherein the plurality of internally hybridized ligation products are detected at one or more FRET signals.

49. The method according to claim 47, wherein the plurality of internally hybridized ligation products with the same FRET signals are distinguished by melting peaks of a first derivative of the melt curve.

50. The method according to claim 49, wherein the same FRET signals from ligation products within the same probe group are distinguished by the melting peaks of the first derivative of the melt curves, which differ by at least 4° C.

51. The method according to claim 49, wherein the same FRET signals from ligation products from the first probe group are distinguished from the FRET signals from ligation products from the second probe group, by the melting peaks of a first derivative of the melt curve, wherein the melting peak for the second group is at least 6° C. higher than for the first group.

52. A kit for identifying one or more of a plurality of target nucleic acid molecules in a sample comprising:
a ligase;
a plurality of oligonucleotide probe sets, each set characterized by (a) a first oligonucleotide probe comprising a target-specific portion and a tunable portion with an endcapped hairpin and (b) a second oligonucleotide probe comprising a target specific portion and a tunable portion, wherein one of the first and second oligonucleotide probes has an acceptor group and the other of the first and second probes has a donor group;

53. A kit according to claim 52 further comprising:
a plurality of oligonucleotide primer sets suitable for amplification of the target nucleic acid molecules and
a polymerase.

54. The kit according to claim 53, wherein each oligonucleotide primer of a primer set comprises a target-portion and a universal tail portion.

55. A method for detecting one or more of a plurality of target nucleic acid molecules in a sample, said method comprising:
providing a sample potentially containing one or more target nucleic acid molecules;
providing a plurality of oligonucleotide probe sets, each set characterized by a first oligonucleotide probe comprising a target-specific portion and a tunable portion with an endcapped hairpin and a second oligonucleotide probe having a target specific portion and a tunable portion, wherein one of the first and second oligonucleotide probes has an acceptor group and the other of the first and second probes has a donor group, and wherein the nucleotide sequence of the tunable portion of the first oligonucleotide probe in a probe set is complementary to the nucleotide sequence of the tunable portion of the second oligonucleotide probe in a probe set;
blending the sample and the plurality of oligonucleotide probe sets to form a hybridization mixture;
subjecting the hybridization mixture to one or more hybridization cycles, each cycle comprising a denaturation treatment, wherein any hybridized nucleic acid sequences are separated, and a hybridization treatment, wherein the target-specific portions of a set of oligonucleotide probes hybridize to their respective target nucleotide sequences, if present in the sample, and the tunable portions of the set of oligonucleotide probes hybridize to each other to form an internally hybridized oligonucleotide probe set; and
detecting the fluorescence resonance energy transfer (FRET) between the donor and acceptor groups of each internally hybridized oligonucleotide probe set, thereby indicating the presence of a target nucleic acid sequences in the sample.

56. The method according to claim 55, wherein the target-specific portions of the oligonucleotides probes in a probe set and the tunable portions of the oligonucleotide probes in a probe set have the same or similar melting temperature.

57. The method according to claim 55 further comprising:
repeating said subjecting and detecting, wherein each time said subjecting is repeated, the hybridization treatment temperature increases.

58. The method according to claim 55 further comprising:
providing a plurality of oligonucleotide primer sets and a DNA polymerase;
blending the sample, the plurality of oligonucleotide primer sets, the plurality of oligonucleotide probe sets, and the DNA polymerase to form a polymerase chain reaction mixture; and
subjecting the polymerase chain reaction mixture to one or more polymerase chain reaction cycles, each cycle comprising a denaturation treatment, wherein any hybridized nucleic acid sequences are separated, a hybridization treatment, wherein the oligonucleotide primer sets hybridize to their respective target nucleotide sequences, if present in the sample, and extend to form extension products and wherein the target-specific portions of a set of oligonucleotide probes hybridize to their respective target nucleotide sequences, if present in the sample, and the tunable portions of the set of oligonucleotide probes hybridize to each other to form an internally hybridized oligonucleotide probe set, wherein said detecting the fluorescence resonance energy transfer (FRET) between the donor and acceptor groups of each internally hybridized oligonucleotide probe set is carried out during each polymerase chain reaction cycle.

59. The method according to claim 58, wherein each primer of the oligonucleotide primer set comprises a target-specific portion and a universal primer pair tail.

60. The method according to claim 58, wherein the plurality of oligonucleotide primer sets form a plurality of oligonucleotide primer groups, each group characterized by oligonucleotide primer sets having the same or similar melting temperature.

61. The method according to claim 58, wherein the plurality of oligonucleotide probe sets form a plurality of oligonucleotide probe groups, each group comprising oligonucleotide probe sets having the same or similar melting temperature.

62. The method according to claim 58 further comprising:
repeating said subjecting and detecting, wherein each time said subjecting is repeated, the denaturation and hybridization treatment temperatures increase.

63. The method according to claim 58, wherein the one or more of a plurality of target nucleic acid molecules with a plurality of sequence differences are present in the sample in unknown amounts, said method further comprising:
providing a known amount of one or more marker target nucleic acid molecules;
providing a plurality of marker-specific oligonucleotide primer sets, each primer of the primer set characterized by a marker-specific target portion and a universal primer tail and
providing a plurality of marker-specific oligonucleotide probe sets, each set characterized by a first oligonucleotide probe comprising a marker target-specific portion and a tunable portion containing an end-capped hairpin and a second oligonucleotide probe having a marker target specific portion and a tunable portion, wherein one of the first and second oligonucleotide probes has an acceptor group and the other of the first and second probes has a donor group, wherein the tunable portions of each probe set have a unique melting temperature, wherein the marker target nucleotide sequences and the plurality of marker-specific oligonucleotide primers sets are mixed with the sample to form the polymerase chain reaction mixture, while the plurality of marker-specific oligonucleotide probe sets are mixed with the plurality of oligonucleotide probe sets to form the hybridization mixture, and wherein said detecting further comprises:
detecting the FRET of the marker-specific oligonucleotide probe sets during each polymerase chain reaction cycle and
comparing the amount of FRET generated from the known amount of marker target nucleotide sequences with the amount of FRET generated from the target nucleotide sequences.

64. The method according to claim 63 further comprising:
quantifying said unknown amounts of target nucleotide sequences based on said comparing.

65. A kit for detecting one or more of a plurality of target nucleic acid molecules in a sample comprising:
a plurality of oligonucleotide probe sets, each set characterized by a first oligonucleotide probe comprising a target-specific portion and a tunable portion with an endcapped hairpin and a second oligonucleotide probe having a target specific portion and a tunable portion, wherein one of the first and second oligonucleotide probes has an acceptor group and the other of the first and second probes has a donor group.

66. The kit according to claim 65 further comprising:
a plurality of oligonucleotide primer sets and a DNA polymerase.

67. The kit according to claim 66, wherein each oligonucleotide primer in a primer set comprises a target-specific portion and a universal tail portion.
